SEQUENCE LISTING (78 sequences)

SEQ ID NO: 1 - DNA, 384 bp, Mus musculus
  source 1..384: mol_type = other DNA
gaggttcagctggagcagcctggggctgaactggtgaagcctggggcttcagtgaagttgtcctgcaaggcttctggctacaccttcaccagctactatatgtactgggtgaagcagaggcctggacaaggccttgagtggattgggggaattaatcctagcaatggtggtactaacttcaatgagaagttcaagaacaaggccacactgactgtagacaaatcctccagcacagcctacatgcaactcagcagcctgacatctgaggactctgcggtctattactgtacaagagagggtaacccctactatactatgaactactggggtcaaggaacctcagtcaccgtctcctcagccaaaacgacacccccatctgtctat

SEQ ID NO: 2 - protein, 128 aa, synthetic construct
EVQLEQPGAELVKPGASVKLSCKASGYTFTSYYMYWVKQRPGQGLEWIGGINPSNGGTNFNEKFKNKATLTVDKSSSTAYMQLSSLTSEDSAVYYCTREGNPYYTMNYWGQGTSVTVSSAKTTPPSVY

SEQ ID NO: 3 - DNA, 324 bp, Mus musculus
  source 1..324: mol_type = other DNA
gatattgtgatgacacagactcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaa

SEQ ID NO: 4 - protein, 108 aa, synthetic construct
DIVMTQTPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 5 - DNA, 384 bp, Mus musculus; identical to SEQ ID NO: 1
  source 1..384: mol_type = other DNA

SEQ ID NO: 6 - protein, 128 aa, synthetic construct; identical to SEQ ID NO: 2

SEQ ID NO: 7 - DNA, 375 bp, Mus musculus
  source 1..375: mol_type = other DNA
gatattgtgatgacccagactccatcctccctgagtgtgtcagcaggagagaaggtcactatgagctgcaagtccagtcagagtctgttaaacagtggaaatcaaaagacctacttggcctggtaccagcagaaaccagggcagcctcctaaactgttgatctacggggcatccactagggaatctggggtccctgatcgcttcacaggcagtggatctggaaccgatttcactcttaccatcagcagtgtgcaggctgaagacctggcagtttattactgtcagaatgatcatagttatccattcacgttcggctcggggacaaagttggaaataaaacgggctgatgctgcaccaactgtatccgcatgcacc

SEQ ID NO: 8 - protein, 122 aa, synthetic construct
DIVMTQTPSSLSVSAGEKVTMSCKSSQSLLNSGNQKTYLAWYQQKPGQPPKLLIYGASTRESGVPDRFTGSGSGTDFTLTISSVQAEDLAVYYCQNDHSYPFTFGSGTKLEIKRADAAPTVS

SEQ ID NO: 9 - DNA, 405 bp, Mus musculus
  source 1..405: mol_type = other DNA
ctggagcagcctggggctgaactggtgaggcctggggcttcagtaatgctgtcctgcaaggcttctggctacatcttcaccacctactggatgcactggctgaagcagaggcctggacaaggccttgactggattggagagattagtcctagcaacggtcgttctaattacaataagaagttcaagagcaaggccacactgactgtagacaaatcctccagcacagcctacatgcaactcagcagcctgacatctgaggactctgcggtctattactgtgcacgattcgacgagggggacttctggggccaaggcaccactctcatagtctcctcagccaaaacaacagccccatcggtctatccactggcccctgtgtgtggagatacaactggctcctcggtg

SEQ ID NO: 10 - protein, 135 aa, synthetic construct
LEQPGAELVRPGASVMLSCKASGYIFTTYWMHWLKQRPGQGLDWIGEISPSNGRSNYNKKFKSKATLTVDKSSSTAYMQLSSLTSEDSAVYYCARFDEGDFWGQGTTLIVSSAKTTAPSVYPLAPVCGDTTGSSV

SEQ ID NO: 11 - DNA, 324 bp, Mus musculus; identical to SEQ ID NO: 3
  source 1..324: mol_type = other DNA

SEQ ID NO: 12 - protein, 108 aa, synthetic construct; identical to SEQ ID NO: 4

SEQ ID NO: 13 - protein, 10 aa, synthetic construct
FLSRPTEKTI

SEQ ID NO: 14 - protein, 10 aa, synthetic construct
KRDPCPHQVD

SEQ ID NO: 15 - protein, 9 aa, synthetic construct
LSAVYTCKR

SEQ ID NO: 16 - DNA, 24 bp, synthetic construct
  source 1..24: mol_type = other DNA
ggctacaccttcaccagctactat

SEQ ID NO: 17 - DNA, 24 bp, synthetic construct
  source 1..24: mol_type = other DNA
attaatcctagcaatggtggtact

SEQ ID NO: 18 - DNA, 36 bp, synthetic construct
  source 1..36: mol_type = other DNA
acaagagagggtaacccctactatactatgaactac

SEQ ID NO: 19 - protein, 8 aa, synthetic construct
GYTFTSYY

SEQ ID NO: 20 - protein, 8 aa, synthetic construct
INPSNGGT

SEQ ID NO: 21 - protein, 12 aa, synthetic construct
TREGNPYYTMNY

SEQ ID NO: 22 - DNA, 24 bp, synthetic construct
  source 1..24: mol_type = other DNA
ggctacatcttcaccacctactgg

SEQ ID NO: 23 - DNA, 24 bp, synthetic construct
  source 1..24: mol_type = other DNA
attagtcctagcaacggtcgttct

SEQ ID NO: 24 - DNA, 24 bp, synthetic construct
  source 1..24: mol_type = other DNA
gcacgattcgacgagggggacttc

SEQ ID NO: 25 - protein, 8 aa, synthetic construct
GYIFITYW

SEQ ID NO: 26 - protein, 8 aa, synthetic construct
ISPSNGRS

SEQ ID NO: 27 - protein, 8 aa, synthetic construct
ARFDEGDF

SEQ ID NO: 28 - DNA, 36 bp, synthetic construct
  source 1..36: mol_type = other DNA
cagagtctgttaaacagtggaaatcaaaagacctac

SEQ ID NO: 29 - skipped

SEQ ID NO: 30 - DNA, 27 bp, synthetic construct
  source 1..27: mol_type = other DNA
cagaatgatcatagttatccattcacg

SEQ ID NO: 31 - protein, 12 aa, synthetic construct
QSLLNSGNQKTY

SEQ ID NO: 32 - skipped

SEQ ID NO: 33 - protein, 9 aa, synthetic construct
QNDYSYPFT

SEQ ID NO: 34 - DNA, 30 bp, synthetic construct
  source 1..30: mol_type = other DNA
aaaagtgtcagtacatctggctatagttat

SEQ ID NO: 35 - skipped

SEQ ID NO: 36 - DNA, 23 bp, synthetic construct
  source 1..23: mol_type = other DNA
cagcacattagggagcttacacg

SEQ ID NO: 37 - protein, 10 aa, synthetic construct
KSVSTSGYSY

SEQ ID NO: 38 - skipped

SEQ ID NO: 39 - protein, 7 aa, synthetic construct
QHIRELT

SEQ ID NO: 40 - DNA, 30 bp, synthetic construct; identical to SEQ ID NO: 34
  source 1..30: mol_type = other DNA

SEQ ID NO: 41 - skipped

SEQ ID NO: 42 - DNA, 24 bp, synthetic construct
  source 1..24: mol_type = other DNA
cagcacattagggagcttacacgt

SEQ ID NO: 43 - protein, 10 aa, synthetic construct
KSVSTSGYSY

SEQ ID NO: 44 - skipped

SEQ ID NO: 45 - protein, 8 aa, synthetic construct
QHIRELTR

SEQ ID NO: 46 - DNA, 31 bp, synthetic construct
  source 1..31: mol_type = other DNA
gagcctcttagaaagcgatggaaagacatat

SEQ ID NO: 47 - skipped

SEQ ID NO: 48 - DNA, 27 bp, synthetic construct
  source 1..27: mol_type = other DNA
tggcaaggtacacattttccgtggacg

SEQ ID NO: 49 - protein, 11 aa, synthetic construct
QSLLESDGKTY

SEQ ID NO: 50 - skipped

SEQ ID NO: 51 - protein, 9 aa, synthetic construct
WQGTHFPWT

SEQ ID NO: 52 - DNA, 357 bp, synthetic construct
  source 1..357: mol_type = other DNA
gaggtccaactccagcagcctggggctgaactggtgaagcctggggcttcagtgaagttgtcctgcaaggcttctggctacaccttcaccagctactatatgtactgggtgaagcagaggcctggacaaggccttgagtggattgggggaattaatcctagcaatggtggtactaacttcaatgagaagttcaagaacaaggccacactgactgtagacaaatcctccagcacagcctacatgcaactcagcagcctgacatctgaggactctgcggtctattactgtacaagagagggtaacccctactatactatgaactactggggtcaaggaacctcagtcaccgtctcctca

SEQ ID NO: 53 - DNA, 345 bp, synthetic construct
  source 1..345: mol_type = other DNA
gaggtccaactccagcaacctggggctgaactggtgaggcctggggcttcagtaatgctgtcctgcaaggcttctggctacatcttcaccacctactggatgcactggctgaagcagaggcctggacaaggccttgactggattggagagattagtcctagcaacggtcgttctaattacaataagaagttcaagagcaaggccacactgactgtagacaaatcctccagcacagcctacatgcaactcagcagcctgacatctgaggactctgcggtctattactgtgcacgattcgacgagggggacttctggggccaaggcaccactctcatagtctcctca

SEQ ID NO: 54 - DNA, 339 bp, synthetic construct
  source 1..339: mol_type = other DNA
gacattgtgatgacgcagtctccatcctccctgagtgtgtcagcaggagagaaggtcactatgagctgcaagtccagtcagagtctgttaaacagtggaaatcaaaagacctacttggcctggtaccagcagaaaccagggcagcctcctaaactgttgatctacggggcatccactagggaatctggggtccctgatcgcttcacaggcagtggatctggaaccgatttcactcttaccatcagcagtgtgcaggctgaagacctggcagtttattactgtcagaatgatcatagttatccattcacgttcggctcggggacaaagttggaaataaaa

SEQ ID NO: 55 - DNA, 330 bp, synthetic construct
  source 1..330: mol_type = other DNA
gacattgtgttgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaatcaaac

SEQ ID NO: 56 - DNA, 330 bp, synthetic construct
  source 1..330: mol_type = other DNA
gatattgtgatgacccagtctcccgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggagggggggaccaagctggaaatcaaa

SEQ ID NO: 57 - DNA, 336 bp, synthetic construct
  source 1..336: mol_type = other DNA
gacgttgtgatgacccagactccactcactttgtcggttaccattggacaaccagcctccatctcttgcaagtcaagtcagagcctcttagaaagcgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtggacgttcggtggaggcaccaagctggaaatcaaa

SEQ ID NO: 58 - protein, 119 aa, synthetic construct
EVQLQQPGAELVKPGASVKLSCKASGYTFTSYYMYWVKQRPGQGLEWIGGINPSNGGTNFNEKFKNKATLTVDKSSSTAYMQLSSLTSEDSAVYYCTREGNPYYTMNYWGQGTSVTVSS

SEQ ID NO: 59 - protein, 114 aa, synthetic construct
EVQLQQPGAELVRPGASVMLSCKASGYIFTTYWMHWLKQRPGQGLDWIGEISPSNGRSNYNKKFKSKATLTVDKSSSTAYMQLSSLTSEDSAVYYCARFDEGDFWGQGTTLIVS

SEQ ID NO: 60 - protein, 112 aa, synthetic construct
DIVMTQSPSSLSVSAGEKVTMSCKSSQSLLNSGNQKTYLAWYQQKPGQPPKLLIYGASTRSGVPDRFTGSGSGTDFTLTISSVQAEDLAVYYCQNDHSYPFTFGSGTKLEIK

SEQ ID NO: 61 - protein, 110 aa, synthetic construct
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWKSN

SEQ ID NO: 62 - protein, 110 aa, synthetic construct
DIVMTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGTKLEIK

SEQ ID NO: 63 - protein, 112 aa, synthetic construct
DVVMTQTPLTLSVTIGQPASISCKSSQSLLESDGKTYLNWLLQRPGQSPKRLIYLVSKLDSGVPDRFTGSGSGTDFTLKISRVEAEDLGVYYCWQGTHFPWTFGGGTKLEIK

SEQ ID NO: 64 - protein, 8 aa, synthetic construct
GYIFITTW

SEQ ID NO: 65 - protein, 9 aa, synthetic construct
QNDHSYPFT

SEQ ID NO: 66 - protein, 8 aa, synthetic construct
INPSNAGT

SEQ ID NO: 67 - protein, 119 aa, synthetic construct
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSS

SEQ ID NO: 68 - protein, 112 aa, synthetic construct
DVVMTQSPLSLPVTIGQPASISCKSSQSLLESDGKTYLNWLQQRPGQSPRRLIYLVSKLDSGVPDRFSGSGSGTDFTLKISRVEAEDVGVYYCWQGTHFPWTFGGGTKVEIK

SEQ ID NO: 69 - protein, 449 aa, synthetic construct
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 447 aa, synthetic construct
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYVVGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - protein, 447 aa, synthetic construct
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYVVGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 72 - protein, 449 aa, synthetic construct
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 449 aa, synthetic construct
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 446 aa, synthetic construct
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - protein, 446 aa, synthetic construct
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 446 aa, synthetic construct
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - protein, 446 aa, synthetic construct
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - protein, 219 aa, synthetic construct
DVVMTQSPLSLPVTIGQPASISCKSSQSLLESDGKTYLNWLQQRPGQSPRRLIYLVSKLDSGVPDRFSGSGSGTDFTLKISRVEAEDVGVYYCWQGTHFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0265369 A1
(43) Pub. Date: Sep. 10, 2026

(54) COMPOSITIONS AND METHODS FOR TREATING OBESITY AND REDUCING FAT ACCUMULATION

(71) Applicant: BOARD OF REGENTS, THE UNIVERSITY OF TEXAS SYSTEM, Austin, TX (US)

(72) Inventors: Jean JIANG, San Antonio, TX (US); Manuel RIQUELME, San Antonio, TX (US); Sumin GU, San Antonio, TX (US); Francisca ACOSTA, San Antonio, TX (US); Jingruo ZHANG, San Antonio, TX (US)

(21) Appl. No.: 19/167,243

(22) PCT Filed: Mar. 22, 2024
(86) PCT No.: PCT/US2024/021167

§ 371 Date: Sep. 19, 2025

Related U.S. Application Data
(60) Provisional application No. 63/454,466, filed on Mar. 24, 2023.

(51) Int. Cl. C07K 16/28; A61K 39/00; A61K 45/06; A61P 3/04; A61P 3/10; A61P 29/00

(52) U.S. Cl. CPC C07K 16/28; A61K 45/06; A61P 3/04; A61P 3/10; A61P 29/00; A61K 2039/505; C07K 2317/24; C07K 2317/54; C07K 2317/55; C07K 2317/565; C07K 2317/75; C07K 2317/76

(57) ABSTRACT

Disclosed herein are antibodies that bind to connexin 43 hemichannels to activate channel opening. Also disclosed herein are methods for treating or preventing obesity, preventing or reducing fat accumulation, reducing weight gain, decreasing bone marrow adiposity, reducing visceral and subcutaneous fat, reducing blood glucose levels, reducing pro-inflammatory macrophage buildup and increasing anti-inflammatory macrophages in visceral adipose tissue, preventing or reducing adipose tissue hypertrophy, and preventing or treating nonalcoholic fatty liver disease or nonalcoholic steatohepatitis with antibodies that activate Cx43 hemichannel opening.

### CROSS REFERENCE TO RELATED APPLICATIONS

This application claims the benefit of the filing date of U.S. Provisional Application No. 63/454,466, filed on Mar. 24, 2023. The content of this earlier filed application is hereby incorporated by reference in its entirety.

### REFERENCE TO A SEQUENCE LISTING

The Sequence Listing submitted herein as an XML file named “21105_0095P1_SL.xml” created on Mar. 22, 2024, and having a size of 73,728 bytes is hereby incorporated by reference pursuant to 37 C.F.R. § 1.52(e)(5).

### BACKGROUND

Overweight and obesity with excessive fat accumulation are associated with health issues and are the third most investigated indications in the metabolic disorders therapy area. Obesity is a significantly preventable, but complex, chronic condition, and a major risk factor for other chronic diseases, including diabetes, musculoskeletal disorders, cardiovascular diseases, and cancers. Although a worldwide epidemic, China and the U.S. outnumber prevalence compared to all other countries combined. Except for dietary and lifestyle changes, no universally effective therapeutics are available for treating obesity. In the current landscape of treatments, efficacy can vary widely, lack long-term success, and have side effects that limit their suitability for certain individuals. Thus, a need exists for treating and preventing obesity.

### SUMMARY OF THE INVENTION

Disclosed herein are methods of treating or preventing obesity in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

Disclosed herein are methods of treating or preventing obesity in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

Disclosed herein are methods of treating or preventing obesity in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

Disclosed herein are methods of preventing or reducing fat accumulation in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

Disclosed herein are methods of preventing or reducing fat accumulation in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

Disclosed herein are methods of preventing or reducing fat accumulation in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

Disclosed herein are methods of preventing or reducing fat accumulation and retaining or increasing lean and/or skeletal muscle mass in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

Disclosed herein are methods of preventing or reducing fat accumulation and retaining or increasing lean and/or skeletal muscle mass in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

Disclosed herein are methods of preventing or reducing fat accumulation and retaining or increasing lean and/or skeletal muscle mass in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

Disclosed herein are methods of reducing weight gain in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

Disclosed herein are methods of reducing weight gain in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

Disclosed herein are methods of reducing weight gain in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

Disclosed herein are methods of reducing weight gain and retaining or increasing lean and/or skeletal muscle mass in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

Disclosed herein are methods of reducing weight gain and retaining or increasing lean and/or skeletal muscle mass in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

Disclosed herein are methods of reducing weight gain and retaining or increasing lean and/or skeletal muscle mass in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

Disclosed herein are methods of decreasing bone marrow adiposity, the methods comprising administering to a subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

Disclosed herein are methods of decreasing bone marrow adiposity, the methods comprising administering to a subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

Disclosed herein are methods of decreasing bone marrow adiposity, the methods comprising administering to a subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

Disclosed herein are methods of reducing the volume of visceral and subcutaneous fat in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

Disclosed herein are methods of reducing the volume of visceral and subcutaneous fat in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

Disclosed herein are methods of reducing the volume of visceral and subcutaneous fat in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

Disclosed herein are methods of reducing the volume of visceral and subcutaneous fat, and retaining or increasing lean and/or skeletal muscle mass in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

Disclosed herein are methods of reducing the volume of visceral and subcutaneous fat, and retaining or increasing lean and/or skeletal muscle mass in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

Disclosed herein are methods of reducing the volume of visceral and subcutaneous fat, and retaining or increasing lean and/or skeletal muscle mass in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

Disclosed herein are methods of reducing blood glucose levels in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

Disclosed herein are methods of reducing blood glucose levels in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

Disclosed herein are methods of reducing blood glucose levels in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

Disclosed herein are methods of reducing pro-inflammatory macrophage buildup in visceral adipose tissue, the methods comprising administering to a subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

Disclosed herein are methods of reducing pro-inflammatory macrophage buildup in visceral adipose tissue, the methods comprising administering to a subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

Disclosed herein are methods of reducing pro-inflammatory macrophage buildup in visceral adipose tissue, the methods comprising administering to a subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

Disclosed herein are methods of increasing anti-inflammatory macrophages in visceral adipose tissue, the methods comprising administering to a subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

Disclosed herein are methods of increasing anti-inflammatory macrophages in visceral adipose tissue, the methods comprising administering to a subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

Disclosed herein are methods of increasing anti-inflammatory macrophages in visceral adipose tissue, the methods comprising administering to a subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

Disclosed herein are methods of preventing or reducing adipose tissue hypertrophy in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

Disclosed herein are methods of preventing or reducing adipose tissue hypertrophy in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

Disclosed herein are methods of preventing or reducing adipose tissue hypertrophy in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

Disclosed herein are methods of preventing or treating nonalcoholic fatty liver disease or nonalcoholic steatohepatitis in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

Disclosed herein are methods of preventing or treating nonalcoholic fatty liver disease or nonalcoholic steatohepatitis in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

Disclosed herein are methods of preventing or treating nonalcoholic fatty liver disease or nonalcoholic steatohepatitis in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

In further embodiments, the anti-connexin Cx43 hemichannel activating antibody or fragments thereof for use according to the embodiments can be any of those described in international (PCT) patent publication no. WO 2015-027120 or WO 2017-147561, which are incorporated herein by reference for their teaching of anti-connexin Cx43 hemichannel activating antibodies, fragment thereof, vectors and cells for making or expressing antibodies.

Disclosed herein are pharmaceutical compositions comprising any of antibodies or fragments thereof as described herein with a pharmaceutically acceptable carrier. Also disclosed herein are antibodies or pharmaceutical compositions for use as a medicament or for use in therapy for treating or preventing obesity, weight gain, and preventing or reducing fat accumulation.

Other aspects of the invention are discussed throughout this application. Any aspect discussed with respect to one aspect of the invention applies to other aspects of the invention as well and vice versa. Each aspect described herein is understood to be aspects of the invention that are applicable to other aspects of the invention. It is contemplated that any aspect discussed herein can be implemented with respect to any method or composition of the invention, and vice versa. Furthermore, compositions and kits of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 shows that injections of an anti-Cx43 hemichannel activating antibody (M2 antibody) decreases bone marrow adiposity. H&E images of bone marrow adiposity (BMA) in the trabecular bone area of aged WT (C57BL/6) male mice (22-months old), subjected to either PBS or M2 antibody (25 mg/kg) intraperitoneal (IP) injections for 2 weeks decreased BMA.

FIG. 2 shows that injections of an anti-Cx43 hemichannel activating antibody (M2 antibody) decreases weight gained as a result of a high fat diet. Weight of diet-induced obesity (DIO) WT (C57BL/6) male mice (16-weeks old, DO or 20-weeks old, D28) subjected to either PBS or M2 antibody (25 mg/kg) intraperitoneal (IP) injections for 4 weeks. DIO mice (60% HFD) n=5, student's t-test, p≤0.05, results presented as mean±SEM.

FIG. 3 shows that an anti-Cx43 hemichannel activating antibody (M2 antibody) treatment significantly decreases percent fat gained as a result of a high fat diet (HFD). Body composition analysis (% fat) of diet-induced obesity (DIO) WT (C57BL/6) male mice (16-weeks old, DO or 20-weeks old, D28) were subjected to either PBS or M2 antibody (25 mg/kg) intraperitoneal (IP) injections for 4 weeks. Percent fat was determined using dual-energy X-ray absorptiometry (DEXA, PIXImus; Lunar, Madison, WI). n=5, student's t-test, p≤0.05, results presented as mean±SEM.

FIG. 4 shows that an anti-Cx43 hemichannel activating antibody (M2 antibody) treatment significantly decreases the volume of visceral and subcutaneous fat. Fat tissue weights (percent normalized to total body weight) of diet-induced obesity (DIO) WT (C57BL/6) male mice (20-weeks old) subjected to either PBS or M2 antibody (25 mg/kg) intraperitoneal (IP) injections for 4 weeks. EPI=epididymal (visceral fat), ING=inguinal (subcutaneous fat), Brown=brown fat. n=5, student's t-test, p≤0.05.

FIG. 5 shows that an anti-Cx43 hemichannel activating antibody (M2 antibody) treatment has a significantly higher brown:white fat ratio, and lower visceral:subcutaneous fat ratio. Fat tissue ratios (normalized to total body weight) of diet-induced obesity (DIO) WT (C57BL/6) male mice (20-weeks old) subjected to either PBS or M2 antibody (25 mg/kg) intraperitoneal (IP) injections for 4 weeks. Brown:white fat ratio=brown (%)/(EPI (%)+ING (%). Visceral:subcutaneous fat ratio=EPI (%)/ING (%). n=5, student's t-test, p≤0.05, results presented as mean±SEM. Higher brown:white fat ratio and lower visceral:subcutaneous fat ratio signifies better metabolic health.

FIG. 6 shows that an anti-Cx43 hemichannel activating antibody (M2 antibody) treatment significantly decreases glucose gained as a result of a high fat diet (HFD). Non-fasted glucose measurements of diet-induced obesity (DIO) WT (C57BL/6) male mice (16-weeks old, DO or 20-weeks old, D28) subjected to either PBS or M2 antibody (25 mg/kg) intraperitoneal (IP) injections for 4 weeks. Glucose was measured using a glucometer (Contour next, Acensia, Parsippany, NJ) using blood collected from the tail vein. n=5, student's t-test, p≤0.05, results presented as mean±SEM.

FIG. 7 shows that an anti-Cx43 hemichannel activating antibody (M2 antibody) treatment decreases pro-inflammatory macrophage buildup and increases anti-inflammatory macrophages in visceral adipose tissue. Adipose tissue macrophage characterization of diet-induced obesity (DIO) WT (C57BL/6) male mice (20-weeks old) subjected to either PBS or M2 antibody (25 mg/kg) intraperitoneal (IP) injections for 4 weeks. Adipose tissue stromal vascular cells (SVCs, containing macrophages) were isolated using collagenase digestion, stained with cell surface markers, and underwent flow cytometry analysis for macrophage characterization. F480+iNOS+ population was used to identify the “M1”, pro-inflammatory phenotype. F480+Arg1+ population was used to identify the “M2”, anti-inflammatory phenotype. EPI=epididymal (visceral fat), ING=inguinal (subcutaneous fat), Brown=brown fat. n=5, student's t-test, p≤0.05, results presented as mean±SEM. M2 antibody injections significantly decreased pro-inflammatory macrophage buildup and increase anti-inflammatory macrophages in visceral adipose tissue, thereby improving adipose tissue metabolic homeostasis.

FIG. 8 shows that an anti-Cx43 hemichannel activating antibody (M2 antibody) treatment decreases M1/M2 macrophage ratio in visceral adipose tissue. Adipose tissue macrophage ratios of diet-induced obesity (DIO) WT (C57BL/6) male mice (20-weeks old) subjected to either PBS or M2 antibody (25 mg/kg) intraperitoneal (IP) injections for 4 weeks. Adipose tissue stromal vascular cells (SVCs, containing macrophages) were isolated using collagenase digestion, stained with cell surface markers, and underwent flow cytometry analysis for macrophage characterization. F480+iNOS+“M1” pro-inflammatory phenotype population (%) was divided by the F480+Arg1+“M2” anti-inflammatory phenotype population (%) to obtain the ratio. EPI=epididymal (visceral fat), ING=inguinal (subcutaneous fat), Brown=brown fat. n=5, student's t-test, p≤0.05 results presented as mean±SEM. Decreases in the M1/M2 ratio in visceral adipose tissue, indicates improved or better metabolic health.

FIG. 9 shows that an anti-Cx43 hemichannel activating antibody (M2 antibody) treatment prevents adipose tissue hypertrophy, NAFLD/NASH, and brown fat white adipose tissue infiltration. H&E images of adipose tissue and liver of diet-induced obesity (DIO) WT (C57BL/6) male mice (20-weeks old) subjected to either PBS or M2 antibody (25 mg/kg) intraperitoneal (IP) injections for 4 weeks. EPI=epididymal (visceral fat), ING=inguinal (subcutaneous fat), Brown=brown fat FIGS. 10A-B show that connexin 43 (Cx43) hemichannels are activated by a humanized anti-Cx43 hemichannel activating antibody (M2 antibody; HmAb2). FIG. 10A shows MLO-Y4 osteocytes were activated by HmAb2, but blocked by a humanized anti-connexin Cx43 hemichannel blocking antibody (M1 antibody; HmAb1). FIG. 10B shows primary mouse osteocytes were activated by HmAb2, but blocked by a humanized anti-connexin Cx43 hemichannel blocking antibody (M1 antibody; HmAb1). Data presented as SEM, and compared to basal control. ***, P<0.001

FIG. 11 shows that an anti-Cx43 hemichannel activating antibody (M2 antibody) treatment significantly decreases percent fat gained as a result of high fat diet (HFD) while helping retain lean/skeletal muscle mass. 60% HFD was maintained throughout the study. n=5, student's t-test, p≤0.05, results presented as mean±SEM.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. If a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosures. Further, the dates of publication provided herein can be different from the actual publication dates, which can require independent confirmation.

### Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. “Optional” or “optionally” means that the subsequently described event, circumstance, or material may or may not occur or be present, and that the description includes instances where the event, circumstance, or material occurs or is present and instances where it does not occur or is not present.

The word “or” as used herein means any one member of a particular list and also includes any combination of members of that list.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

“Inhibit,” “inhibiting” and “inhibition” mean to diminish or decrease an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

“Treatment” and “treating” refer to administration or application of a therapeutic agent (e.g., an anti-Cx43 hemichannel activating antibody described herein) to a subject or performance of a procedure or modality on a subject for the purpose of obtaining a therapeutic benefit of a disease or health-related condition. For example, a treatment may include administration of a pharmaceutically effective amount of an antibody that enhances or stimulates the opening of the Cx43 hemichannel. In some aspects, the anti-Cx43 hemichannel activating antibody that binds to a connexin 43 (Cx43) hemichannel and enhances channel opening can have no effect on gap channel coupling.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be obesity, diabetes, NASH or NAFLD.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment, such as, for example, prior to the administering step.

The term “fragment” can refer to a portion (e.g., at least 5, 10, 25, 50, 100, 125, 150, 200, 250, 300, 350, 400, or 500, etc. amino acids or nucleic acids) of a protein or nucleic acid molecule that is substantially identical to a reference protein or nucleic acid and retains the biological activity of the reference. In some aspects, the fragment or portion retains at least 50%, 75%, 80%, 85%, 90%, 95%, or 99% of the biological activity of the reference protein or nucleic acid described herein. Further, a fragment of a referenced peptide can be a continuous or contiguous portion of the referenced polypeptide (e.g., a fragment of a peptide that is ten amino acids long can be any 2-9 contiguous residues within that peptide).

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions are those within the following groups: Ser, Thr, and Cys; Leu, Be, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, they may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂(CH₂)ₙCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

As used herein, “nonalcoholic fatty liver disease” and “NAFLD” can be used interchangeably with “metabolic dysfunction-associated steatotic liver disease” and “MASLD”, respectively.

As used herein, “nonalcoholic steatohepatitis” and “NASH” can be used interchangeably with “metabolic dysfunction-associated steatohepatitis” and “MASH”, respectively.

As used herein, the term “MIH” refers to an antibody that was cloned from hybridoma clones. “M1” refers to hybridoma monoclonal 1, and “H” refers to the variable heavy chain.

As used herein, the term “MIM7K” refers to the variable light chain identified from hybridoma clones M1 and M7.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that can be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state.

Moieties of the invention, such as polypeptides, peptides, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.”

The term “providing” is used according to its ordinary meaning “to supply or furnish for use.” In some embodiments, the protein is provided directly by administering the protein, while in other embodiments, the protein is effectively provided by administering a nucleic acid that encodes the protein. In certain aspects the invention contemplates compositions comprising various combinations of nucleic acid, antigens, peptides, and/or epitopes.

The phrase “specifically binds” or “specifically immunoreactive” to a target refers to a binding reaction that is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein the terms “amino acid” and “amino acid identity” refers to one of the 20 naturally occurring amino acids or any non-natural analogues that may be in any of the antibodies, variants, or fragments disclosed. Thus “amino acid” as used herein means both naturally occurring and synthetic amino acids. For example, homophenylalanine, citrulline and norleucine are considered amino acids for the purposes of the invention. “Amino acid” also includes amino acid residues such as proline and hydroxyproline. The side chain may be in either the (R) or the (S) configuration. In some aspects, the amino acids are in the (S) or L-configuration. If non-naturally occurring side chains are used, non-amino acid substituents may be used, for example to prevent or retard in vivo degradation.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Various cells are able to communicate with each other and with the extracellular environment through hemichannels and gap junctions formed by the protein connexin. Connexin proteins are ubiquitously expressed throughout the body. Six connexin proteins make up one hemichannel, and two hemichannels make up one gap junction channel. Gap junctions are a cluster of channels that are located in the plasma membrane between adjoining cells and they mediate intercellular communication. Hemichannels are a separate entity from gap junction channels. Hemichannels permit the exchange of molecules between the intracellular compartments and the extracellular environment.

Disclosed herein are compositions and methods using an anti-Cx43 hemichannel activating antibody that can reduce white fat tissue mass, reduce the size of adipose in both white and brown fat tissues. Thus, the compositions and methods disclosed herein are useful in treating and preventing obesity and type II diabetes.

Disclosed herein are methods of identifying reagents that modulate the opening of connexin hemichannels. In some aspects, modulate can mean stimulate or enhance the opening of one or more connexin hemichannels. In some aspects, the connexin hemichannel can be a Cx 43 hemichannel. In some aspects, the methods can identify compounds or drugs that positively modulate (i.e., stimulate or enhance) the opening of connexin hemichannels. Other embodiments are directed to methods of treating obesity, diabetes, NASH, or NAFLD by administering a compound that open connexin 43 hemichannels or stimulate or enhance the opening of connexin 43 hemichannels to a patient diagnosed with or having or at risk for obesity, diabetes, NASH, or NAFLD. In some aspects, the compounds that open Cx43 hemichannels or stimulate or enhance the opening of Cx43 hemichannels can be used to prevent or reduce fat accumulation in a subject, reduce weight gain in a subject, decrease bone marrow adiposity, reduce the volume of visceral and subcutaneous fat in a subject, reduce blood glucose levels, reducing pro-inflammatory macrophage buildup in visceral adipose tissue, increase anti-inflammatory macrophages in visceral adipose tissue, or prevent or reduce adipose hypertrophy in a subject. In some aspects, compounds that open Cx43 channels or stimulate or enhance the opening of Cx43 hemichannels can be used to treat obesity, diabetes, NASH, or NAFLD. In some aspects, the antibody that binds to a connexin 43 (Cx43) hemichannel and enhances channel opening can have no effect on gap channel coupling.

### Antibodies

Disclosed herein are antibodies that can stimulate or enhance the opening hemichannels, and in particular, Cx43 hemichannels. In some aspects, the antibody that binds to a connexin 43 (Cx43) hemichannel and enhances channel opening can have no effect on gap channel coupling. As used herein, an anti-Cx43 hemichannel activating antibody that stimulates or enhances the opening of Cx43 hemichannels can be referred to as “M2 antibody”. An example of identifying and isolating a monoclonal antibody is described below.

The term “CDR” as used herein refers to a Complementarity Determining Region of an antibody variable domain. Systematic identification of residues included in the CDRs have been developed by Kabat et al. (1991, Sequences of Proteins of Immunological Interest, 5th Ed., United States Public Health Service, National Institutes of Health, Bethesda). Variable light chain (VL) CDRs are herein defined to include residues at positions 27-32 (CDR1), 50-56 (CDR2), and 91-97 (CDR3). Variable heavy chain (VH) CDRs are herein defined to include residues at positions 27-33 (CDR1), 52-56 (CDR2), and 95-102 (CDR3).

The CDRs disclosed herein may also include variants. Generally, the amino acid identity between individual variant CDRs is at least 90%, 91%, 92%, 93%, 94%, 95%, 96%, 97%, 98%, 99%, or 100%. Thus, a “variant CDR” is one with the specified identity to the parent or reference CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. For example, a “variant CDR” can be a sequence that contains 1, 2, 3 or 4 amino acid changes as compared to the parent or reference CDR of the invention, and shares or improves biological function, specificity and/or activity of the parent CDR.

Disclosed herein are anti-Cx43 hemichannel activating antibodies comprising a first VH CDR corresponding to SEQ ID NO: 19, a second VH CDR corresponding to SEQ ID NO: 20, a third VH CDR corresponding to SEQ ID NO: 21, a first VL CDR corresponding to SEQ ID NO: 49, a second VL CDR corresponding to SEQ ID NO: 50, and a third VL CDR corresponding to SEQ ID NO: 51. In some aspects, the anti-Cx43 hemichannel activating antibody can be a humanized antibody. In some aspects, the anti-Cx43 hemichannel activating antibody can be an IgG, IgM, IgA, IgD, IgE, or a genetically modified IgG class antibody comprising a first VH CDR corresponding to SEQ ID NO: 19, a second VH CDR corresponding to SEQ ID NO: 20, a third VH CDR corresponding to SEQ ID NO: 21, a first VL CDR corresponding to SEQ ID NO: 49, a second VL CDR corresponding to SEQ ID NO: 50, and a third VL CDR corresponding to SEQ ID NO: 51. In some aspects, the anti-Cx43 hemichannel activating antibody can be an IgG class of antibody, wherein the IgG class antibody can be an IgG1, IgG2, IgG3, or IgG4 class antibody. In some aspects, the anti-Cx43 hemichannel activating antibody can comprise a VH amino acid sequence at least 90% identical to SEQ ID NO: 58 and/or a VL amino acid sequence at least 90% identical to SEQ ID NO: 63. In some aspects, the anti-Cx43 hemichannel activating antibody comprises a VH amino acid sequence according to SEQ ID NO: 58 and/or a VL amino acid sequence according to SEQ ID NO: 63.

Disclosed herein are recombinant anti-Cx43 hemichannel activating antibodies or fragments thereof. In some aspects, recombinant the anti-Cx43 hemichannel activating antibody comprises a first VH CDR corresponding to SEQ ID NO: 19 or a fragment thereof, a second VH CDR corresponding to SEQ ID NO: 20 or a fragment thereof, a third VH CDR corresponding to SEQ ID NO: 21 or a fragment thereof, a first VL CDR corresponding to SEQ ID NO: 49 or a fragment thereof, a second VL CDR corresponding to SEQ ID NO: 50 or a fragment thereof, and a third VL CDR corresponding to SEQ ID NO: 51 or a fragment thereof. In some aspects, the recombinant anti-Cx43 hemichannel activating antibody or fragment thereof can be a humanized antibody. In some aspects, the recombinant anti-Cx43 hemichannel activating antibody can be an IgG, IgM, IgA, IgD, IgE, or a genetically modified IgG class antibody comprising a first VH CDR corresponding to SEQ ID NO: 19, a second VH CDR corresponding to SEQ ID NO: 20, a third VH CDR corresponding to SEQ ID NO: 21, a first VL CDR corresponding to SEQ ID NO: 49, a second VL CDR corresponding to SEQ ID NO: 50, and a third VL CDR corresponding to SEQ ID NO: 51. In some aspects, the recombinant anti-Cx43 hemichannel activating antibody can be an IgG class of antibody, wherein the IgG class antibody can be an IgG1, IgG2, IgG3, or IgG4 class antibody. In some aspects, the recombinant anti-Cx43 hemichannel activating antibody comprises a VH amino acid sequence at least 90% identical to SEQ ID NO: 58 or a fragment thereof and/or a VL amino acid sequence at least 90% identical to SEQ ID NO: 63 or a fragment thereof. In some aspects, the recombinant anti-Cx43 hemichannel activating antibody can comprise a VH amino acid sequence according to SEQ ID NO: 58 or a fragment thereof and/or a VL amino acid sequence according to SEQ ID NO: 63 or a fragment thereof.

Disclosed herein are anti-Cx43 hemichannel activating antibodies directed against hemichannel polypeptides, and nucleic acid molecules encoding said anti-Cx43 hemichannel activating antibodies. In some aspects, the anti-Cx43 hemichannel activating antibody can bind an epitope having an amino acid sequence of FLSRPTEKTI (SEQ ID NO: 13), KRDPCPHQVD (SEQ ID NO: 14), or LSAVYTCKR (SEQ ID NO: 15). In some aspects, the anti-Cx43 hemichannel activating antibody can bind an epitope having an amino acid sequence of FLSRPTEKTI (SEQ ID NO: 13).

In some aspects, a first heavy chain region can comprise an amino acid sequence having an amino acid sequence of residues 13 to 37 of SEQ ID NO: 2; a second heavy chain region having an amino acid sequence corresponding to residues 46 to 66 of SEQ ID NO: 2; and a third heavy chain region comprising an amino acid sequence having an amino acid sequence of residues 97 to 116 of SEQ ID NO: 2. SEQ ID NO: 2: EVQLEQPGAELVKPGASVKLSCKASGYTFTSYYMYWVKQRPGQGLEWIGGINPSNG GTNFNEKFKNKATLTVDKSSSTAYMQLSSLTSEDSAVYYCTREGNPYYTMNYWGQ GTSVTVSSAKTTPPSVY.

In some aspects, the anti-Cx43 hemichannel activating antibodies disclosed herein can include full-length antibodies, antibody fragments, single chain antibodies, bispecific antibodies, minibodies, domain antibodies, synthetic antibodies and antibody fusions, and fragments thereof.

As used herein, the term “antigen” is a molecule capable of being bound by an antibody or T-cell receptor. In some aspects, binding moieties other than antibodies can be engineered to specifically bind to an antigen, e.g., aptamers, avimers, and the like.

The term “antibody” or “immunoglobulin” is used to include intact antibodies and binding fragments/segments thereof. As used herein, the term “antibody” is intended to refer broadly to any immunologic binding agent, such as IgG, IgM, IgA, IgD, IgE, and genetically modified IgG as well as polypeptides comprising antibody CDR domains that retain antigen binding activity. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand. Typically, fragments compete with the intact antibody from which they were derived for specific binding to an antigen. Fragments include separate heavy chains, light chains, Fab, Fab′ F(ab′)2, Fabc, and Fv. Fragments/segments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibodies. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai and Lachmann, Clin Exp Immunol 79:315-21, 1990; Kostelny et al., J. Immunol. 148:1547-53, 1992.

The term “antibody” can include five different classes of human immunoglobulins, namely IgG, IgA, IgM, IgD, and IgE. In some aspects, the disclosed antibodies can be an IgG class of antibody which can be classified into the 4 subclasses of IgG1, IgG2, IgG3, and IgG4. In some aspects, the disclosed antibodies can be an IgA class of antibody which, can be classified into the 2 subclasses of IgA1 and IgA2. The basic structure of immunoglobulin is made up of 2 homologous L chains (light chains) and 2 homologous H chains (heavy chains). The immunoglobulin class and subclass are determined by H chains. In some aspects, the antibody or antibodies or variants or fragments thereof can be an IgG4.

While antigen-binding specificity is maintained, antibody stability of IgG4 can be improved. In some aspects, the antibody can be improved, for example, by substituting arginine (R) of IgG4 with glutamic acid (E), phenylalanine (F), isoleucine (I), asparagine (N), glutamine (Q), serine (S), valine (V), tryptophan (W), tyrosine (Y), lysine (K), threonine (T), methionine (M), or leucine (L).

In some aspects, any of CDR sequences disclosed herein can include a single amino acid change as compared to the parent or reference CDR. In some aspects, any of the CDR sequences disclosed herein can include at least two amino acid changes as compared to the parent or reference CDR. In some aspects, the amino acid change can be a change from a cysteine residue to another amino acid. In some aspects, the amino acid change can be a change from a glycine residue to another amino acid. In some aspects, the at least one amino acid change or substitution can decrease deamidation. The amino acid identity between individual variant CDRs can be at least 60%, 65%, 70%, 75%, 80%, 85%, 90%, 91%, 92%, 93%, 94%, 95%, 96%, 97%, 98%, 99% or 100%. Thus, a “variant CDR” can be one with the specified identity to the parent CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. For example, the parent CDR sequence can be one or more of SEQ ID NOs: 19, 20, 21, 49, 50, and/or 51. The variant CDR sequence can be at least 60%, 65%, 70%, 75%, 80%, 85%, 90%, 91%, 92%, 93%, 94%, 95%, 96%, 97%, 98%, 99% or 100% identical to any one of SEQ ID NOs: 19, 20, 21, 49, 50, and/or 51. The variant CDR sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

As discussed herein, minor variations in the amino acid sequences of any of the anti-Cx43 hemichannel activating antibodies disclosed herein are contemplated as being encompassed by the instant disclosure, providing that the variations in the amino acid sequence maintains at least 75%, more preferably at least 80%, 90%, 95%, and most preferably 99% sequence identity to the parent sequence. In some aspects, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are known to one of ordinary skill in the art.

In some aspects, amino acid substitutions can be those which: (1) reduce susceptibility to proteolysis, (2) reduce susceptibility to oxidation, (3) alter binding affinity for forming protein complexes, (4) alter binding affinities, (5) reduces or decreases deamidation; and (6) confer or modify other physiocochemical or functional properties of such analogs. In some aspects, single or multiple amino acid substitutions (preferably conservative amino acid substitutions) may be made in the non-CDR sequence of the heavy chain, the light chain or both. In some aspects, one or more amino acid substitutions can be made in one or more of the CDR sequences of the heavy chain, the light chain or both.

In some aspects, the at least one amino acid change can be to substitute an NG motif (amino acid asparagine followed by a glycine). In some aspects, the glycine residue can be substituted or replaced with hydrophobic amino acid residue. In some aspects, the glycine residue can be substituted or replaced with alanine, aspartic acid, glutamic acid, or valine. In some aspects, the glycine residue can be substituted or replaced with arginine, lysine, or glutamine.

Many methods have been developed for chemical labeling and enhancement of the properties of antibodies and their common fragments, including the Fab and F(ab′)2 fragments. Somewhat selective reduction of some antibody disulfide bonds has been previously achieved, yielding antibodies and antibody fragments that can be labeled at defined sites, enhancing their utility and properties. Selective reduction of the two hinge disulfide bonds present in F(ab′)2 fragments using mild reduction has been useful. In some aspects, cysteine and methionine can be susceptible to rapid oxidation, which can negatively influence the cleavage of protecting groups during synthesis and the subsequent peptide purification. In some instances, cysteine residues in peptides used for antibody production can affect the avidity of the antibody, because free cysteines are uncommon in vivo and therefore may not be recognized by the native peptide structure. In some aspects, the disclosed antibodies and fragments thereof comprise a sequence where a cysteine reside outside of the CDR (e.g., in the non-CDR sequence of the heavy chain, the light chain or both) is substituted. In some aspects, cysteine can be replaced with serine and methionine replaced with norleucine (Nle). Multiple cysteines on a peptide or in one of the disclosed antibodies or fragments thereof may be susceptible to forming disulfide linkages unless a reducing agent such as dithiothreitol (DTT) is added to the buffer or the cysteines can be replaced with serine residues.

While the site or region for introducing an amino acid sequence variation is predetermined, the mutation per se need not be predetermined. For example, in order to optimize the performance of a mutation at a given site, random mutagenesis may be conducted at the target codon or region and the expressed antigen binding protein CDR variants screened for the optimal combination of desired activity. Techniques for making substitution mutations at predetermined sites in DNA having a known sequence are well known, for example, M13 primer mutagenesis and PCR mutagenesis. Screening of the mutants is done using assays of antigen binding protein activities as described herein.

Amino acid substitutions are typically of single residues; insertions usually will be on the order of from about one (1) to about twenty (20) amino acid residues, although considerably larger insertions may be tolerated. Deletions range from about one (1) to about twenty (20) amino acid residues, although in some cases deletions may be much larger.

Substitutions, deletions, insertions or any combination thereof may be used to arrive at a final derivative or variant. Generally these changes are done on a few amino acids to minimize the alteration of the molecule, particularly the immunogenicity and specificity of the antigen binding protein. However, larger changes may be tolerated in certain circumstances.

A “fragment antigen-binding fragment (Fab)” is a region of an antibody that binds to antigen. A Fab comprises constant and variable regions from both heavy and light chains. By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein, or any other antibody embodiments as outlined herein.

By “Fv” or “Fv fragment” or “Fv region” as used herein is meant a polypeptide that comprises the VL and VH domains of a single antibody.

A “CDR” or complementarity determining region is a region of hypervariability interspersed within regions that are more conserved, termed “framework regions” (FR). By “framework” as used herein is meant the region of an antibody variable domain exclusive of those regions defined as CDRs. Each antibody variable domain framework can be further subdivided into the contiguous regions separated by the CDRs (FR1, FR2, FR3 and FR4).

The term “antigen-binding portion” of an antibody (or simply “antibody portion”), as used herein, refers to one or more fragments of an antibody that retain the ability to specifically bind to an antigen (e.g., hemichannel). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL/VK, VH, CL and CH1 domains; (ii) a F(ab′)2 fragment, a bivalent fragment comprising two Fab fragments linked by a disulfide bridge at the hinge region; (iii) a Fab′ fragment, which can be an Fab with part of the hinge region (see, Fundamental Immunology (Paul ed., 3rd ed. 1993); (iv) a Fd fragment consisting of the VH and CH1 domains; (v) a Fv fragment consisting of the VL and VH domains of a single arm of an antibody; (vi) a dAb fragment (Ward et al., (1989) Nature 341:544-546), which consists of a VH domain; (vii) an isolated complementarity determining region (CDR); and (viii) a nanobody, a heavy chain variable region containing a single variable domain and two constant domains.

The term “specifically binds” (or “immunospecifically binds”) is not intended to indicate that an antibody binds exclusively to its intended target. Rather, an antibody “specifically binds” if its affinity for its intended target is about 5-fold greater when compared to its affinity for a non-target molecule. Suitably there is no significant cross-reaction or cross-binding with undesired substances. The affinity of the antibody will, for example, be at least about 5-fold, such as 10-fold, such as 25-fold, especially 50-fold, and particularly 100-fold or more, greater for a target molecule than its affinity for a non-target molecule. In some aspects, specific binding between an antibody or other binding agent and an antigen means a binding affinity of at least 10⁶ M⁻¹. Antibodies may, for example, bind with affinities of at least about 10⁷ M⁻¹, such as between about 10⁸ M⁻¹ to about 10⁹ M⁻¹, about 10⁹ M⁻¹ to about 10¹⁰ M⁻¹, or about 10⁻¹⁰M⁻¹ to about 10¹¹ M⁻¹. Antibodies may, for example, bind with an EC50 of 50 nM or less, 10 nM or less, 1 nM or less, 100 pM or less, or more preferably 10 pM or less. In some aspects, the antibodies can bind with an EC50 of about 60 μg/ml, 59 μg/ml, 58 μg/ml, 57 μg/ml, 56 μg/ml, 55 μg/ml, 54 μg/ml, 53 μg/ml, 52 μg/ml, 51 μg/ml, 50 μg/ml or less. In some aspects, the antibodies can bind with an EC50 of about 50 μg/ml, 49 μg/ml, 48 μg/ml, 47 μg/ml, 46 μg/ml, 45 μg/ml, 44 μg/ml, 43 μg/ml, 42 μg/ml, 41 μg/ml, 40 μg/ml or less. In some aspects, the antibodies can bind with an EC50 of about 40 μg/ml, 39 μg/ml, 38 μg/ml, 37 μg/ml, 36 μg/ml, 35 μg/ml, 34 μg/ml, 33 μg/ml, 32 μg/ml, 31 μg/ml, 30 μg/ml or less.

In some aspects, the anti-Cx43 hemichannel activating antibodies described herein can specifically bind to their intended target. In some aspects, the anti-Cx43 hemichannel activating antibody antibodies described herein have no off site binding. For example, the anti-Cx43 hemichannel activating antibody antibodies described herein do not bind or are not distributed to the heart, liver or spinal cord.

The anti-Cx43 hemichannel activating antibodies described herein can be variants including, without limitation, a fragment (e.g., an Fab fragment or an F(ab′)2 fragment of, e.g., a tetrameric antibody), a fragment of an scFv or diabody, or a variant of a tetrameric antibody, an scFv, a diabody, or fragments thereof that differ by virtue of the addition and/or substitution of one or more amino acid residues. The antibody moiety can be further engineered as, for example, a di-diabody.

As is well known in the art, certain types of antibody fragments can be generated by enzymatic treatment of a “full-length” antibody. Digestion with papain produces two identical Fab fragments, each with a single antigen-binding site, and a residual Fc fragment. The Fab fragment also contains the constant domain of the light chain and the Chi domain of the heavy chain. In contrast, digestion with pepsin yields the F(ab′)2 fragment that has two antigen-binding sites and is still capable of cross-linking antigen.

Fab′ fragments differ from Fab fragments in that they include additional residues at the C-terminus of the Chi domain, including one or more cysteine residues from the antibody hinge region. The cysteine residues of the constant domains bear a free thiol group. F(ab′)2 antibody fragments are pairs of Fab′ fragments linked by cysteine residues in the hinge region. Other chemical couplings of antibody fragments are also known in the art.

The Fv region is a minimal fragment that contains a complete antigen-recognition and binding site consisting of one heavy chain and one light chain variable domain. The three CDRs of each variable domain interact to define an antigen-biding site on the surface of the VH-VL dimer. Collectively, the six CDRs confer antigen-binding specificity to the antibody.

A “single-chain variable fragment (scFv)” means a protein comprising the variable regions of the heavy and light chains of an antibody. As would be known in the art, a “single-chain” antibody or “scFv” fragment is a single chain Fv variant formed when the VH and VL domains of an antibody are included in a single polypeptide chain that recognizes and binds an antigen. Typically, single-chain antibodies include a polypeptide linker between the VH and VL domains that allows the scFv to form a desired three-dimensional structure for antigen binding (see, e.g., Pluckthun, In The Pharmacology of Monoclonal Antibodies, Rosenburg and Moore Eds., Springer-Verlag, New York, 113:269-315. 1994). For example, a scFv can be a fusion protein comprising a variable heavy chain, a linker, and a variable light chain. In some aspects, the linker can be a short, flexible fragment that can be about 8 to 20 amino acids in length. For example, (G4S)n can be used (n=1, 2, 3 or 4).

In some aspects, the anti-Cx43 hemichannel activating antibody can be a diabody. Diabodies are small antibody fragments that have two antigen-binding sites. Each fragment contains a VH domain concatenated to a VL domain. However, since the linker between the domains is too short to allow pairing between them on the same chain, the linked Vh-Vl domains are forced to pair with complementary domains of another chain, creating two antigen-binding sites. Diabodies are described more fully, for example, in EP 404,097; WO 93/11161; and Hollinger et al., Proc. Natl. Acad. Sci. USA 90:6444-6448, 1993.

In some aspects, an anti-Cx43 hemichannel activating antibody or a fragment thereof that binds to at least a portion of Cx43 protein and stimulates, promotes or enhances the opening of Cx43 hemichannels and stimulates or enhances signaling and prevents or treats obesity, prevents or treats diabetes, prevents or treats NASH, or NAFLD, prevents or reduces fat accumulation, reduces weight gain, decreases bone marrow adiposity, reduces the volume of visceral and subcutaneous fat, reduces blood glucose levels, reduces pro-inflammatory macrophage buildup in visceral adipose tissue, increases anti-inflammatory macrophages in visceral adipose tissue, or prevents or reduces adipose hypertrophy are contemplated. In some aspects, an anti-Cx43 hemichannel activating antibody or a fragment thereof that binds to at least a portion of Cx43 protein and stimulates, promotes or enhances the opening of Cx43 hemichannels and stimulates or enhances signaling and prevents or treats obesity, prevents or treats diabetes, prevents or treats NASH, or NAFLD, prevents or reduces fat accumulation, reduces weight gain, decreases bone marrow adiposity, reduces the volume of visceral and subcutaneous fat, reduces blood glucose levels, reduces pro-inflammatory macrophage buildup in visceral adipose tissue, increases anti-inflammatory macrophages in visceral adipose tissue, retains and/or increases lean and/or skeletal muscle mass with fat loss, or prevents or reduces adipose hypertrophy and has no effect on gap junction coupling are contemplated. In some aspects, the anti-Cx43 hemichannel activating antibody can be a monoclonal antibody, polyclonal antibody or a humanized antibody. Thus, by known means and as described herein, polyclonal or monoclonal antibodies, antibody fragments, and binding domains and CDRs (including engineered forms of any of the foregoing) may be created that are specific to Cx43 protein, one or more of its respective epitopes, or conjugates of any of the foregoing, whether such antigens or epitopes are isolated from natural sources or are synthetic derivatives or variants of the natural compounds.

Examples of antibody fragments suitable include without limitation: (i) the Fab fragment, consisting of VL, VH, CL, and CH1 domains; (ii) the “Fd” fragment consisting of the VII and Cm domains; (iii) the “Fv” fragment consisting of the VL and VH domains of a single antibody; (iv) the “dAb” fragment, which consists of a VH domain; (v) isolated CDR regions; (vi) F(ab′)2 fragments, a bivalent fragment comprising two linked Fab fragments; (vii) single chain Fv molecules (“scFv”), wherein a VII domain and a VL domain are linked by a peptide linker that allows the two domains to associate to form a binding domain; (viii) bispecific single chain Fv dimers (see U.S. Pat. No. 5,091,513); and (ix) diabodies, multivalent or multispecific fragments constructed by gene fusion (US Patent App. Pub. 20050214860). Fv, scFv, or diabody molecules may be stabilized by the incorporation of disulfide bridges linking the VH and VL domains. Minibodies comprising a scFv joined to a CH3 domain may also be made (Hu et al., 1996).

Antibody-like binding peptidomimetics are also contemplated. Liu et al. (2003) describe “antibody like binding peptidomimetics” (ABiPs), which are peptides that act as pared-down antibodies and have certain advantages of longer serum half-life as well as less cumbersome synthesis methods.

Animals may be inoculated with an antigen, such as a Cx43 extracellular domain protein, in order to produce antibodies specific for Cx43 protein. Frequently an antigen is bound or conjugated to another molecule to enhance the immune response. As used herein, a conjugate is any peptide, polypeptide, protein, or non-proteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes. A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

The term “monoclonal antibody” (monoclonal antibody) refers to an antibody, or population of like anti-bodies, obtained from a population of substantially homogeneous antibodies, and is not to be construed as requiring production of the antibody by any particular method, including but not limited to, monoclonal antibodies can be made by the hybridoma method first described by Kohler and Milstein (Nature, 256: 495-497, 1975), or by recombinant DNA methods. A monoclonal antibody is a single species of antibody wherein every antibody molecule recognizes the same epitope because the antibody producing cells are derived from a single B-lymphocyte cell line. The methods for generating monoclonal antibodies (MAbs) generally begin along the same lines as those for preparing polyclonal antibodies. In some aspects, rodents such as mice and rats are used in generating monoclonal antibodies. In some aspects, rabbit, sheep, or frog cells are used in generating monoclonal antibodies. The use of rats is well known and may provide certain advantages. Mice (e.g., BALB/c mice) are routinely used and generally give a high percentage of stable fusions.

Hybridoma technology involves the fusion of a single B lymphocyte from a mouse previously immunized with a Cx43 antigen with an immortal myeloma cell (usually mouse myeloma). This technology provides a method to propagate a single antibody-producing cell for an indefinite number of generations, such that unlimited quantities of structurally identical antibodies having the same antigen or epitope specificity (monoclonal antibodies) may be produced.

Plasma B cells may be isolated from freshly prepared rabbit peripheral blood mononuclear cells of immunized rabbits and further selected for Cx43 binding cells. After enrichment of antibody producing B cells, total RNA may be isolated and cDNA synthesized. DNA sequences of antibody variable regions from both heavy chains and light chains may be amplified, constructed into a phage display Fab expression vector, and transformed into E. coli. Cx43 specific binding Fab may be selected out through multiple rounds enrichment panning and sequenced. Selected Cx43 binding hits may be expressed as full length IgG in rabbit and rabbit/human chimeric forms using a mammalian expression vector system in human embryonic kidney (HEK293) cells (Invitrogen) and purified using a protein G resin with a fast protein liquid chromatography (FPLC) separation unit.

In some aspects, the anti-Cx43 hemichannel activating antibody can be a chimeric antibody, for example, an antibody comprising antigen binding sequences from a non-human donor grafted to a heterologous non-human, human, or humanized sequence (e.g., framework and/or constant domain sequences). The term “chimeric antibody” (or “chimeric immunoglobulin”) refers to a molecule comprising a heavy and/or light chain which is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly et al. (1984), infra; Morrison et al., Proc. Natl. Acad. Sci. U.S.A. 81:6851).

Methods have been developed to replace light and heavy chain constant domains of the monoclonal antibody with analogous domains of human origin, leaving the variable regions of the foreign antibody intact. Alternatively, “fully human” monoclonal antibodies can be produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024. All patents, patent application publications, and other publications cited herein and therein are hereby incorporated by reference in the present application.

Antibodies may be produced from any animal source, including birds and mammals. Preferably, the antibodies are ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference. These techniques are further described in: Marks (1992); Stemmer (1994); Gram et al. (1992); Barbas et al. (1994); and Schier et al. (1996).

It is fully expected that the anti-Cx43 hemichannel activating antibodies disclosed herein will have effects on Cx43 hemichannels regardless of the animal species, monoclonal cell line, or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into antibody fragments having the binding domain or CDR. Removal of the Fc portion reduces the likelihood that the antigen antibody fragment will elicit an undesirable immunological response, and thus, antibodies without Fc may be preferential for prophylactic or therapeutic treatments. As described herein, antibodies may also be constructed so as to be chimeric or partially or fully human, so as to reduce or eliminate the adverse immunological consequences resulting from administering to an animal an antibody that has been produced in, or has sequences from, other species.

The term “humanized antibody” refers to forms of antibodies that contain sequences from non-human (e.g., murine) antibodies as well as human antibodies. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. Furthermore, humanized antibodies can comprise residues that are found neither in the recipient antibody nor in the imported CDR or framework sequences. These modifications are made to further refine and maximize antibody performance. Thus, in general, a humanized antibody can comprise all or substantially all of at least one, and in one aspect two, variable domains, in which all or substantially all of the hypervariable loops correspond to those of a non-human immunoglobulin and all or substantially all of the FR regions are those of a human immunoglobulin sequence. The humanized antibody optionally also can comprise at least a portion of an immunoglobulin constant region (Fc), or that of a human immunoglobulin (see, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276B1; Winter, U.S. Pat. No. 5,225,539; Winter, European Patent No. 0,239,400 B1; Padlan, E. A. et al., European Patent Application No. 0,519,596 A1; Queen et al. (1989) Proc. Natl. Acad. Sci. USA, Vol 86:10029-10033).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that a bacteria containing such a variant may be implemented in compositions and methods. Consequently, a protein need not be isolated.

It is contemplated that in compositions there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be an antibody that binds Cx43.

An antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

Also described herein are antibodies (e.g., an anti-Cx43 hemichannel activating antibody) and antibody-like molecules against Cx43, polypeptides and peptides that are linked to at least one agent to form an antibody conjugate or payload. In order to increase the efficacy of antibody molecules as diagnostic or therapeutic agents, to the antibody can be linked or covalently bound or complexed to at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, therapeutic enzymes, antibiotics, radio-labeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody. Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

In some aspects, the anti-Cx43 hemichannel activating antibody described herein can comprise a heavy chain immunoglobulin variable region comprising complementarity determining region 1 (CDR1) comprising the sequence of SEQ ID NO: 19; CDR2 comprising the sequence of SEQ ID NO: 20 or SEQ ID NO: 66; and a CDR3 comprising the sequence of SEQ ID NO: 21. Table 3 shows examples of CDRs of the heavy chain.

In some aspects, the anti-Cx43 hemichannel activating antibody described herein can comprise a light chain immunoglobulin variable region comprising complementarity determining region 1 (CDR1) comprising the sequence of SEQ ID NO: 49; CDR2 comprising the sequence of SEQ ID NO: 50; and a CDR3 comprising the sequence of SEQ ID NO: 51. Table 3 shows examples of CDRs in the light chain.

In some aspects, the anti-Cx43 hemichannel activating antibody described herein can comprise a heavy chain immunoglobulin variable region comprising complementarity determining region 1 (CDR1) comprising the sequence of SEQ ID NO: 19; CDR2 comprising the sequence of SEQ ID NO: 20 or SEQ ID NO: 66; and a CDR3 comprising the sequence of SEQ ID NO: 21; and a light chain immunoglobulin variable region comprising complementarity determining region 1 (CDR1) comprising the sequence of SEQ ID NO: 49; CDR2 comprising the sequence of SEQ ID NO: 50; and a CDR3 comprising the sequence of SEQ ID NO: 51.

In some aspects, the anti-Cx43 hemichannel activating antibody described herein can comprise a variable heavy chain comprising a sequence having at least 90% identity to a sequence set forth in SEQ ID NOs: 19, 20, or 21 (see, Table 3). In some aspects, the anti-Cx43 hemichannel activating antibody described herein comprises a variable heavy chain comprising a sequence having at least 90, 91, 92, 93, 94, 95, 96, 97, 98, 99, or 100% identity to a sequence set forth in SEQ ID NOs: 19, 20 (or SEQ ID NO: 66), or 21.

In some aspects, the anti-Cx43 hemichannel activating antibody described herein can comprise a variable light chain comprising a sequence having at least 90% identity to a sequence set forth in SEQ ID NOs: 49, 50 or 51 (see, Table 3). In some aspects, the anti-Cx43 hemichannel activating antibody described herein comprises a variable light chain comprising a sequence having at least 90, 91, 92, 93, 94, 95, 96, 97, 98, 99, or 100% identity to a sequence set forth in SEQ ID NOs: 49, 50 or 51.

Disclosed herein are nucleic acid sequences that encode M1H comprising the sequence of SEQ ID NO: 52. Disclosed herein are nucleic acid sequences that encode M1M7K comprising the sequence of SEQ ID NO: 57.

Disclosed herein are nucleic acid sequences encoding M1H comprising a variable heavy chain comprising a sequence having at least 90% identity to a sequence set forth in SEQ ID NO: 52 (see, Table 4). In some aspects, M1H comprises a variable heavy chain comprising a sequence having at least 90, 91, 92, 93, 94, 95, 96, 97, 98, 99 or 100% identity to a sequence set forth in SEQ ID NO: 52.

Disclosed herein are nucleic acid sequences encoding M1M7K comprising a variable light chain comprising a sequence having at least 90% identity to a sequence set forth in SEQ ID NO: 57 (see, Table 4). In some aspects, M1M7K comprises a variable light chain comprising a sequence having at least 90, 91, 92, 93, 94, 95, 96, 97, 98, 99 or 100% identity to a sequence set forth in SEQ ID NO: 57.

Disclosed herein are nucleic acid sequences that encode the M1H region comprising a heavy chain immunoglobulin variable region comprising a CDR1 comprising the sequence of SEQ ID NO: 16; a CDR2 comprising a comprising the sequence of SEQ ID NO: 17; a CDR3 comprising a comprising the sequence of SEQ ID NO: 18.

Disclosed herein are nucleic acid sequences that encode the M1M7K region comprising a light chain immunoglobulin variable region comprising a CDR1 comprising the sequence of SEQ ID NO: 46; a CDR2 comprising a comprising the sequence of SEQ ID NO: 47; a CDR3 comprising a comprising the sequence of SEQ ID NO: 48.

Disclosed herein are nucleic acid sequences that encode the anti-Cx43 hemichannel activating antibody comprising a heavy chain immunoglobulin variable region comprising a CDR1 comprising the sequence of SEQ ID NO: 16; a CDR2 comprising a comprising the sequence of SEQ ID NO: 17; a CDR3 comprising a comprising the sequence of SEQ ID NO: 18; and a light chain immunoglobulin variable region comprising a CDR1 comprising the sequence of SEQ ID NO: 46; a CDR2 comprising a comprising the sequence of SEQ ID NO: 47; a CDR3 comprising a comprising the sequence of SEQ ID NO: 48.

Disclosed herein are antibodies or fragments thereof that bind to human Cx43. Also described herein are anti-Cx43 hemichannel activating antibodies. In some aspects, the anti-Cx43 hemichannel activating antibody or fragment thereof comprises a variable heavy chain comprising a sequence having at least 90% identity to one of the variable heavy chain amino acid sequences provided in Tables 2 or 4. In some aspects, the anti-Cx43 hemichannel activating antibody or fragment thereof comprises a variable heavy chain comprising a sequence having at least 90% identity to a sequence set forth in SEQ ID NO: 58. In some aspects, the anti-Cx43 hemichannel activating antibody or fragment thereof comprises a variable heavy chain comprising a sequence set forth in SEQ ID NO: 58.

Disclosed herein are antibodies or fragments thereof that bind to human Cx43. Also disclosed herein are anti-Cx43 hemichannel activating antibodies. In some aspects, the anti-Cx43 hemichannel activating antibody or fragment thereof comprises a variable light chain comprising a sequence having at least 90% identity to one of the variable light chain amino acid sequences provided in Tables 2 or 4. In some aspects, the anti-Cx43 hemichannel activating antibody or fragment thereof comprises a variable light chain comprising a sequence having at least 90% identity to a sequence set forth in SEQ ID NO: 63. In some aspects, the anti-Cx43 hemichannel activating antibody or fragment thereof comprises a variable light chain comprising a sequence set forth in SEQ ID NOs: 63.

Disclosed herein are antibodies or fragments thereof that bind to human Cx-43 hemichannels. Also disclosed herein are anti-Cx43 hemichannel activating antibodies. In some aspects, the anti-Cx43 hemichannel activating antibody or fragment thereof comprises a variable heavy chain comprising a sequence having at least 90% identity to a sequence set forth in SEQ ID NO: 58, and a variable light chain comprising a sequence having at least 90% identity to a sequence set forth in SEQ ID NO: 63. In some aspects, the anti-Cx43 hemichannel activating antibody comprises a variable heavy chain comprising a sequence set forth in SEQ ID NO: 58 and a variable light chain comprising a sequence set forth in SEQ ID NO: 63.

In some aspects, the anti-Cx43 hemichannel activating antibody or fragment thereof comprises a M1H region. In some aspects, the M1H region comprises a heavy chain immunoglobulin variable region comprising a CDR1 comprising the sequence of SEQ ID NO: 19; a CDR2 comprising the sequence of SEQ ID NO: 20; and a CDR3 comprising the sequence of SEQ ID NO: 21.

In some aspects, the anti-Cx43 hemichannel activating antibody or fragment thereof comprises a M1M7K region. In some aspects, the M1M7K region comprises a light chain immunoglobulin variable region comprising a CDR1 comprising the sequence of SEQ ID NO: 49; a CDR2 comprising the sequence of 50; and a CDR3 comprising the sequence of SEQ ID NO: 51.

In some instances, the disclosed anti-Cx43 hemichannel activating antibodies or fragments thereof further comprise a tag sequence.

Disclosed herein are nucleic acid sequences that encode the disclosed antibodies (e.g., anti-Cx43 hemichannel activating antibodies) or fragments thereof. For example, disclosed are nucleic acid sequences comprising a variable heavy chain comprising a sequence having at least 90% identity to a sequence set forth in SEQ ID NO: 52. Disclosed herein are nucleic acid sequences that encode the disclosed antibodies (e.g., anti-Cx43 hemichannel activating antibodies) or fragments thereof. For example, disclosed herein are nucleic acid sequences comprising a variable heavy chain comprising a sequence set forth in SEQ ID NO: 52. Also disclosed herein are nucleic acid sequences comprising a variable light chain comprising a sequence having at least 90% identity to a sequence set forth in SEQ ID NO: 57. Also disclosed are nucleic acid sequences comprising a variable light chain comprising a sequence set forth in SEQ ID NO: 57.

Disclosed herein are nucleic acid sequences comprising a variable heavy chain comprising a sequence having at least 90% identity to a sequence set forth in SEQ ID NO: 52; and a variable light chain comprising a sequence having at least 90% identity a sequence set forth in SEQ ID NO: 57. Disclosed are nucleic acid sequences comprising a variable heavy chain comprising a sequence set forth in SEQ ID NO: 52; and a variable light chain comprising a sequence set forth in SEQ ID NO: 57.

Disclosed herein are nucleic acid sequences capable of encoding a single chain variable fragment comprising a variable heavy chain comprising a sequence having at least 90% identity a sequence set forth in SEQ ID NO: 58.

Disclosed are nucleic acid sequences capable of encoding a single chain variable fragment comprising a variable light chain comprising a sequence having at least 90% identity a sequence set forth in SEQ ID NO: 63.

Disclosed are nucleic acid sequences capable of encoding a single chain variable fragment comprising a variable heavy chain comprising a sequence having at least 90% identity a sequence set forth in SEQ ID NO:58; and a variable light chain comprising a sequence having at least 90% identity a sequence set forth in SEQ ID NO:63.

In some instances, the disclosed antibodies (e.g., anti-Cx43 hemichannel activating antibodies) or fragments thereof can be bispecific. For example, the anti-Cx43 hemichannel activating antibody or fragment thereof can comprise a first Fab region comprising the heavy and light chain of SEQ ID NO: 58 and a second Fab region comprising the heavy and light chain of SEQ ID NO: 63, wherein the first and second Fab regions can be different.

In some instances, the bispecific antibodies can be trifunctional.

In some instances, the disclosed antibodies (e.g., anti-Cx43 hemichannel activating antibodies) or fragments thereof can be mouse, human, humanized, chimeric, or a combination thereof.

In some instances, the disclosed antibodies (e.g., anti-Cx43 hemichannel activating antibodies) or fragments thereof are monoclonal.

### Methods

Disclosed herein are methods of treating or preventing obesity in a subject with anti-Cx43 hemichannel activating antibodies or fragments thereof. Disclosed herein are methods of preventing or reducing fat accumulation in a subject with anti-Cx43 hemichannel activating antibodies or fragments thereof. Disclosed herein are methods of reducing weight gain in a subject with anti-Cx43 hemichannel activating antibodies or fragments thereof. Disclosed herein are methods of decreasing bone marrow adiposity in a subject with anti-Cx43 hemichannel activating antibodies or fragments thereof. Disclosed herein are methods of reducing the volume of visceral and subcutaneous fat in a subject with anti-Cx43 hemichannel activating antibodies or fragments thereof. Disclosed herein are methods of reducing blood glucose levels in a subject with anti-Cx43 hemichannel activating antibodies or fragments thereof. Disclosed herein are methods of reducing pro-inflammatory macrophage buildup in visceral adipose tissue with anti-Cx43 hemichannel activating antibodies or fragments thereof. Disclosed herein are methods of increasing anti-inflammatory macrophages in visceral adipose tissue with anti-Cx43 hemichannel activating antibodies or fragments thereof. Disclosed herein are methods of preventing or reducing adipose tissue hypertrophy in a subject with anti-Cx43 hemichannel activating antibodies or fragments thereof. Disclosed herein are methods of preventing or treating nonalcoholic fatty liver disease or nonalcoholic steatohepatitis in a subject with anti-Cx43 hemichannel activating antibodies or fragments thereof. Disclosed herein are methods of preventing or reducing fat accumulation and retaining or increasing lean and/or skeletal muscle mass in a subject. Disclosed herein are methods of reducing weight gain and retaining or increasing lean and/or skeletal muscle mass in a subject. Disclosed herein are methods of reducing the volume of visceral and subcutaneous fat, and retaining or increasing lean and/or skeletal muscle mass in a subject.

In some aspects, the methods disclosed herein can comprise administering to the subject a therapeutically effective amount of an anti-Cx43 hemichannel activating antibody or fragment thereof. In some aspects, the anti-Cx43 hemichannel activating antibody or fragment thereof can comprise a variable heavy chain comprising a sequence having at least 90% identity to a sequence set forth in SEQ ID NO: 58. In some aspects, the anti-Cx43 hemichannel activating antibody or fragment thereof can comprise a variable light chain comprising a sequence having at least 90% identity to a sequence set forth in SEQ ID NO: 63. In some aspects, the anti-Cx43 hemichannel activating antibody or fragment thereof can comprise a variable heavy chain comprising a sequence having at least 90% identity to a sequence set forth in SEQ ID NO: 58 and a variable light chain comprising a sequence having at least 90% identity to a sequence set forth in SEQ ID NO: 63.

In some aspects, the anti-Cx43 hemichannel activating antibody or fragment thereof can comprise: a heavy chain immunoglobulin variable region comprising: a first complementarity determining region 1 comprising a sequence having at least 60% identity to SEQ ID NO: 19; a second complementarity determining region 2 comprising a sequence having at least 60% identity to SEQ ID NO: 20; and a third complementarity determining region 3 comprising a sequence having at least 60% identity to SEQ ID NO: 21. In some aspects, the anti-Cx43 hemichannel activating antibody or fragment thereof can comprise: a light chain immunoglobulin variable region comprising: a first complementarity determining region 1 comprising a sequence having at least 60% identity to SEQ ID NO: 49; a second complementarity determining region 2 comprising a sequence having at least 60% identity to SEQ ID NO: 50; and a third complementarity determining region 3 comprising a sequence having at least 60% identity to SEQ ID NO: 51.

In some aspects, the anti-Cx43 hemichannel activating antibody or fragment thereof can comprise: a heavy chain immunoglobulin variable region comprising: a first complementarity determining region 1 comprising a sequence having a single amino acid change compared to SEQ ID NO: 19; a second complementarity determining region 2 comprising a sequence a single amino acid change compared to SEQ ID NO: 20; and a third complementarity determining region 3 comprising a sequence a single amino acid change compared to SEQ ID NO: 21. In some aspects, the anti-Cx43 hemichannel activating antibody or fragment thereof can comprise: a light chain immunoglobulin variable determining region comprising: a first complementarity determining region 1 comprising a sequence having a single amino acid change compared to SEQ ID NO: 49; a second complementarity determining region 2 comprising a sequence having a single amino acid change compared to SEQ ID NO: 50; and a third complementarity determining region 3 comprising a sequence having a single amino acid change compared to SEQ ID NO: 51.

In some aspects, the anti-Cx43 hemichannel activating antibody or fragment thereof can comprise a heavy chain immunoglobulin variable region comprising: a) a complementarity determining region 1 (CDR1) comprising the sequence of SEQ ID NO: 19 or a variant thereof, b) a complementarity determining region 2 (CDR2) comprising the sequence of SEQ ID NO: 20 or a variant thereof (e.g., SEQ ID NO: 66); and/or c) a complementarity determining region (CDR3) comprising the sequence of SEQ ID NO: 21 or a variant thereof. In some aspects, any one of the heavy chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid. In some aspects, the at least one amino acid substitution can decrease or reduce deamidation.

In some aspects, the anti-Cx43 hemichannel activating antibody or fragment thereof can comprise a light chain immunoglobulin variable region comprising: a) a complementarity determining region 1 (CDR1) comprising the sequence of SEQ ID NO: 49 or a variant thereof, b) a complementarity determining region 2 (CDR2) comprising the sequence of SEQ ID NO: 50 or a variant thereof, and/or c) a complementarity determining region 3 (CDR3) comprising the sequence of SEQ ID NO: 51 or a variant thereof. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid. In some aspects, the other amino acid can be an alanine. In some aspects, the at least one amino acid substitution can decrease or reduce deamidation.

In some aspects, the anti-Cx43 hemichannel activating antibody or fragment thereof can comprise a heavy chain immunoglobulin variable region comprising: a complementarity determining region 1 (CDR1) comprising a sequence having at least 60% identity to a sequence set forth in SEQ ID NO: 19; a complementarity determining region 2 (CDR2) comprising a sequence having at least 60% identity to a sequence set forth in SEQ ID NO: 20; and/or a complementarity determining region 3 (CDR3) comprising a sequence having at least 60% identity to a sequence set forth in SEQ ID NO: 21. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid. In some aspects, the other amino acid can be an alanine. In some aspects, the at least one amino acid substitution can decrease or reduce deamidation. For example, the at least one amino acid substitution can be a glycine residue to an alanine residue. In some aspects, the antibody or fragment thereof can comprise a heavy chain immunoglobulin variable region comprising a CDR2 comprising a sequence set forth in SEQ ID NO: 66 (INPSNAGT).

In some aspects, the anti-Cx43 hemichannel activating antibody or fragment thereof can comprise a light chain immunoglobulin variable region comprising: a complementarity determining region 1 (CDR1) comprising a sequence having at least 60% identity to a sequence set forth in SEQ ID NO: 49; a complementarity determining region 2 (CDR2) comprising a sequence having at least 60% identity to a sequence set forth in SEQ ID NO: 50; and/or a complementarity determining region 3 (CDR3) comprising a sequence having at least 60% identity to a sequence set forth in SEQ ID NO: 51. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid. In some aspects, the other amino acid can be an alanine. In some aspects, the at least one amino acid substitution can decrease or reduce deamidation.

In some aspects, the anti-Cx43 hemichannel activating antibody or fragment thereof can comprise a heavy chain immunoglobulin variable region comprising: a complementarity determining region 1 (CDR1) comprising a sequence having a single amino acid change compared to a sequence set forth in SEQ ID NO: 19; a complementarity determining region 2 (CDR2) comprising a sequence having a single amino acid change compared to a sequence set forth in SEQ ID NO: 20; and/or a complementarity determining region 3 (CDR3) comprising a sequence having a single amino acid change compared to a sequence set forth in SEQ ID NO: 21. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid. In some aspects, the other amino acid can be an alanine. In some aspects, the at least one amino acid substitution can decrease or reduce deamidation. For example, the at least one amino acid substitution can be a glycine residue to an alanine residue. In some aspects, the antibody or fragment thereof can comprise a heavy chain immunoglobulin variable region comprising a CDR2 comprising a sequence set forth in SEQ ID NO: 66 (INPSNAGT).

In some aspects, the anti-Cx43 hemichannel activating antibody or fragment thereof can comprise a light chain immunoglobulin variable region comprising: a complementarity determining region 1 (CDR1) comprising a sequence having a single amino acid change compared to a sequence set forth in SEQ ID NO: 49; a complementarity determining region 2 (CDR2) comprising a sequence having a single amino acid change compared to a sequence set forth in SEQ ID NO: 50; and/or a complementarity determining region 3 (CDR3) comprising a sequence having a single amino acid change compared to a sequence set forth in SEQ ID NO: 51. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid. In some aspects, the other amino acid can be an alanine. In some aspects, the at least one amino acid substitution can decrease or reduce deamidation.

In some aspects, any of the methods disclosed herein can comprise administering to the subject an effective amount of an expression vector encoding the anti-Cx43 hemichannel activating antibody or fragment thereof. In some aspects, the anti-Cx43 hemichannel activating antibody or fragment thereof can be administered in a pharmaceutically acceptable composition. In some aspects, the pharmaceutical composition can be lyophilized. In some aspects, the anti-Cx43 hemichannel activating antibody or fragment thereof can be administered systemically. In some aspects, the anti-Cx43 hemichannel activating antibody or fragment thereof can be administered intravenously, intradermally, intratumorally, intramuscularly, intraperitoneally, subcutaneously, or locally. In some aspects, the anti-Cx43 hemichannel activating antibody or fragment thereof can be a humanized antibody or humanized fragment thereof. In some aspects, the anti-Cx43 hemichannel activating antibody can be an IgG, IgM, IgA, IgD, IgE, or a genetically modified IgG class antibody comprising a first VH CDR corresponding to SEQ ID NO: 19, a second VH CDR corresponding to SEQ ID NO: 20 (or SEQ ID NO: 66), a third VH CDR corresponding to SEQ ID NO: 21, a first VL CDR corresponding to SEQ ID NO: 49, a second VL CDR corresponding to SEQ ID NO: 50, and a third VL CDR corresponding to SEQ ID NO: 51. In some aspects, the antibody can be an IgG class of antibody, wherein the IgG class antibody is an IgG1, IgG2, IgG3, or IgG4 class antibody.

Disclosed herein are methods of treating or preventing obesity in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising the sequence of SEQ ID NO: 19; b) a complementarity determining region 2 (CDR2) comprising the sequence of SEQ ID NO: 20; and/or c) a complementarity determining region (CDR3) comprising the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising the sequence of SEQ ID NO: 49; b) a complementarity determining region 2 (CDR2) comprising the sequence of SEQ ID NO: 50; and/or c) a complementarity determining region 3 (CDR3) comprising the sequence of SEQ ID NO: 51. In some aspects, the methods of treating or preventing obesity in a subject further comprise retaining or increasing lean and/or skeletal muscle mass in the subject.

Disclosed herein are methods of treating or preventing obesity in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising a variant of the sequence of SEQ ID NO: 19; b) a complementarity determining region 2 (CDR2) comprising a variant of the sequence of SEQ ID NO: 20; and/or c) a complementarity determining region (CDR3) comprising a variant of the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising a variant of the sequence of SEQ ID NO: 49; b) a complementarity determining region 2 (CDR2) comprising a variant of the sequence of SEQ ID NO: 50; and/or c) a complementarity determining region 3 (CDR3) comprising a variant of the sequence of SEQ ID NO: 51, wherein any one of the heavy chain CDR1, CDR2 or CDR3 variants or the light chain CDR1, CDR2 or CDR variants comprise at least one amino acid substitution as compared to the parent CDR. In some aspects, the methods of treating or preventing obesity in a subject further comprise retaining or increasing lean and/or skeletal muscle mass in the subject.

Disclosed herein are methods of preventing or reducing fat accumulation in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising the sequence of SEQ ID NO: 19; b) a complementarity determining region 2 (CDR2) comprising the sequence of SEQ ID NO: 20; and/or c) a complementarity determining region (CDR3) comprising the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising the sequence of SEQ ID NO: 49; b) a complementarity determining region 2 (CDR2) comprising the sequence of SEQ ID NO: 50; and/or c) a complementarity determining region 3 (CDR3) comprising the sequence of SEQ ID NO: 51. In some aspects, the methods of preventing or reducing fat accumulation in a subject further comprise retaining or increasing lean and/or skeletal muscle mass in the subject.

Disclosed herein are methods of preventing or reducing fat accumulation in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising a variant of the sequence of SEQ ID NO: 19; b) a complementarity determining region 2 (CDR2) comprising a variant of the sequence of SEQ ID NO: 20; and/or c) a complementarity determining region (CDR3) comprising a variant of the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising a variant of the sequence of SEQ ID NO: 49; b) a complementarity determining region 2 (CDR2) comprising a variant of the sequence of SEQ ID NO: 50; and/or c) a complementarity determining region 3 (CDR3) comprising a variant of the sequence of SEQ ID NO: 51, wherein any one of the heavy chain CDR1, CDR2 or CDR3 variants or the light chain CDR1, CDR2 or CDR variants comprise at least one amino acid substitution as compared to the parent CDR. In some aspects, the methods of preventing or reducing fat accumulation in a subject further comprise retaining or increasing lean and/or skeletal muscle mass in the subject.

Disclosed herein are methods of preventing or reducing fat accumulation and retaining or increasing lean and/or skeletal muscle mass in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising the sequence of SEQ ID NO: 19; b) a complementarity determining region 2 (CDR2) comprising the sequence of SEQ ID NO: 20; and/or c) a complementarity determining region (CDR3) comprising the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising the sequence of SEQ ID NO: 49; b) a complementarity determining region 2 (CDR2) comprising the sequence of SEQ ID NO: 50; and/or c) a complementarity determining region 3 (CDR3) comprising the sequence of SEQ ID NO: 51.

Disclosed herein are methods of preventing or reducing fat accumulation and retaining or increasing lean and/or skeletal muscle mass in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising a variant of the sequence of SEQ ID NO: 19; b) a complementarity determining region 2 (CDR2) comprising a variant of the sequence of SEQ ID NO: 20; and/or c) a complementarity determining region (CDR3) comprising a variant of the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising a variant of the sequence of SEQ ID NO: 49; b) a complementarity determining region 2 (CDR2) comprising a variant of the sequence of SEQ ID NO: 50; and/or c) a complementarity determining region 3 (CDR3) comprising a variant of the sequence of SEQ ID NO: 51, wherein any one of the heavy chain CDR1, CDR2 or CDR3 variants or the light chain CDR1, CDR2 or CDR variants comprise at least one amino acid substitution as compared to the parent CDR.

Disclosed herein are methods of reducing weight gain in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising the sequence of SEQ ID NO: 19; b) a complementarity determining region 2 (CDR2) comprising the sequence of SEQ ID NO: 20; and/or c) a complementarity determining region (CDR3) comprising the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising the sequence of SEQ ID NO: 49; b) a complementarity determining region 2 (CDR2) comprising the sequence of SEQ ID NO: 50; and/or c) a complementarity determining region 3 (CDR3) comprising the sequence of SEQ ID NO: 51. In some aspects, methods of reducing weight gain in a subject further comprise retaining or increasing lean and/or skeletal muscle mass in the subject.

Disclosed herein are methods of reducing weight gain in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising a variant of the sequence of SEQ ID NO: 19; b) a complementarity determining region 2 (CDR2) comprising a variant of the sequence of SEQ ID NO: 20; and/or c) a complementarity determining region (CDR3) comprising a variant of the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising a variant of the sequence of SEQ ID NO: 49; b) a complementarity determining region 2 (CDR2) comprising a variant of the sequence of SEQ ID NO: 50; and/or c) a complementarity determining region 3 (CDR3) comprising a variant of the sequence of SEQ ID NO: 51, wherein any one of the heavy chain CDR1, CDR2 or CDR3 variants or the light chain CDR1, CDR2 or CDR variants comprise at least one amino acid substitution as compared to the parent CDR. In some aspects, methods of reducing weight gain in a subject further comprise retaining or increasing lean and/or skeletal muscle mass in the subject.

Disclosed herein are methods of reducing weight gain and retaining or increasing lean and/or skeletal muscle mass in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising the sequence of SEQ ID NO: 19; b) a complementarity determining region 2 (CDR2) comprising the sequence of SEQ ID NO: 20; and/or c) a complementarity determining region (CDR3) comprising the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising the sequence of SEQ ID NO: 49; b) a complementarity determining region 2 (CDR2) comprising the sequence of SEQ ID NO: 50; and/or c) a complementarity determining region 3 (CDR3) comprising the sequence of SEQ ID NO: 51.

Disclosed herein are methods of reducing weight gain and retaining or increasing lean and/or skeletal muscle mass in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising a variant of the sequence of SEQ ID NO: 19; b) a complementarity determining region 2 (CDR2) comprising a variant of the sequence of SEQ ID NO: 20; and/or c) a complementarity determining region (CDR3) comprising a variant of the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising a variant of the sequence of SEQ ID NO: 49; b) a complementarity determining region 2 (CDR2) comprising a variant of the sequence of SEQ ID NO: 50; and/or c) a complementarity determining region 3 (CDR3) comprising a variant of the sequence of SEQ ID NO: 51, wherein any one of the heavy chain CDR1, CDR2 or CDR3 variants or the light chain CDR1, CDR2 or CDR variants comprise at least one amino acid substitution as compared to the parent CDR.

Disclosed herein are methods of decreasing bone marrow adiposity, the methods comprising administering to a subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising the sequence of SEQ ID NO: 19; b) a complementarity determining region 2 (CDR2) comprising the sequence of SEQ ID NO: 20; and/or c) a complementarity determining region (CDR3) comprising the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising the sequence of SEQ ID NO: 49; b) a complementarity determining region 2 (CDR2) comprising the sequence of SEQ ID NO: 50; and/or c) a complementarity determining region 3 (CDR3) comprising the sequence of SEQ ID NO: 51. In some aspects, the methods of decreasing bone marrow adiposity further comprise retaining or increasing lean and/or skeletal muscle mass in the subject.

Disclosed herein are methods of decreasing bone marrow adiposity, the methods comprising administering to a subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising a variant of the sequence of SEQ ID NO: 19; b) a complementarity determining region 2 (CDR2) comprising a variant of the sequence of SEQ ID NO: 20; and/or c) a complementarity determining region (CDR3) comprising a variant of the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising a variant of the sequence of SEQ ID NO: 49; b) a complementarity determining region 2 (CDR2) comprising a variant of the sequence of SEQ ID NO: 50; and/or c) a complementarity determining region 3 (CDR3) comprising a variant of the sequence of SEQ ID NO: 51, wherein any one of the heavy chain CDR1, CDR2 or CDR3 variants or the light chain CDR1, CDR2 or CDR variants comprise at least one amino acid substitution as compared to the parent CDR. In some aspects, the methods of decreasing bone marrow adiposity further comprise retaining or increasing lean and/or skeletal muscle mass in the subject.

Disclosed herein are methods of reducing the volume of visceral and subcutaneous fat in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising the sequence of SEQ ID NO: 19; b) a complementarity determining region 2 (CDR2) comprising the sequence of SEQ ID NO: 20; and/or c) a complementarity determining region (CDR3) comprising the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising the sequence of SEQ ID NO: 49; b) a complementarity determining region 2 (CDR2) comprising the sequence of SEQ ID NO: 50; and/or c) a complementarity determining region 3 (CDR3) comprising the sequence of SEQ ID NO: 51. In some aspects, the methods of reducing the volume of visceral and subcutaneous fat in a subject further comprise retaining or increasing lean and/or skeletal muscle mass in the subject.

Disclosed herein are methods of reducing the volume of visceral and subcutaneous fat in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising a variant of the sequence of SEQ ID NO: 19; b) a complementarity determining region 2 (CDR2) comprising a variant of the sequence of SEQ ID NO: 20; and/or c) a complementarity determining region (CDR3) comprising a variant of the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising a variant of the sequence of SEQ ID NO: 49; b) a complementarity determining region 2 (CDR2) comprising a variant of the sequence of SEQ ID NO: 50; and/or c) a complementarity determining region 3 (CDR3) comprising a variant of the sequence of SEQ ID NO: 51, wherein any one of the heavy chain CDR1, CDR2 or CDR3 variants or the light chain CDR1, CDR2 or CDR variants comprise at least one amino acid substitution as compared to the parent CDR. In some aspects, the methods of reducing the volume of visceral and subcutaneous fat in a subject further comprise retaining or increasing lean and/or skeletal muscle mass in the subject.

Disclosed herein are methods of reducing the volume of visceral and subcutaneous fat, and retaining or increasing lean and/or skeletal muscle mass in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising the sequence of SEQ ID NO: 19; b) a complementarity determining region 2 (CDR2) comprising the sequence of SEQ ID NO: 20; and/or c) a complementarity determining region (CDR3) comprising the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising the sequence of SEQ ID NO: 49; b) a complementarity determining region 2 (CDR2) comprising the sequence of SEQ ID NO: 50; and/or c) a complementarity determining region 3 (CDR3) comprising the sequence of SEQ ID NO: 51.

Disclosed herein are methods of reducing the volume of visceral and subcutaneous fat, and retaining or increasing lean and/or skeletal muscle mass in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising a variant of the sequence of SEQ ID NO: 19; b) a complementarity determining region 2 (CDR2) comprising a variant of the sequence of SEQ ID NO: 20; and/or c) a complementarity determining region (CDR3) comprising a variant of the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising a variant of the sequence of SEQ ID NO: 49; b) a complementarity determining region 2 (CDR2) comprising a variant of the sequence of SEQ ID NO: 50; and/or c) a complementarity determining region 3 (CDR3) comprising a variant of the sequence of SEQ ID NO: 51, wherein any one of the heavy chain CDR1, CDR2 or CDR3 variants or the light chain CDR1, CDR2 or CDR variants comprise at least one amino acid substitution as compared to the parent CDR.

Disclosed herein are methods of reducing blood glucose levels in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising the sequence of SEQ ID NO: 19; b) a complementarity determining region 2 (CDR2) comprising the sequence of SEQ ID NO: 20; and/or c) a complementarity determining region (CDR3) comprising the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising the sequence of SEQ ID NO: 49; b) a complementarity determining region 2 (CDR2) comprising the sequence of SEQ ID NO: 50; and/or c) a complementarity determining region 3 (CDR3) comprising the sequence of SEQ ID NO: 51. In some aspects, the methods of reducing blood glucose levels in a subject further comprise retaining or increasing lean and/or skeletal muscle mass in the subject.

Disclosed herein are reducing blood glucose levels in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising a variant of the sequence of SEQ ID NO: 19; b) a complementarity determining region 2 (CDR2) comprising a variant of the sequence of SEQ ID NO: 20; and/or c) a complementarity determining region (CDR3) comprising a variant of the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising a variant of the sequence of SEQ ID NO: 49; b) a complementarity determining region 2 (CDR2) comprising a variant of the sequence of SEQ ID NO: 50; and/or c) a complementarity determining region 3 (CDR3) comprising a variant of the sequence of SEQ ID NO: 51, wherein any one of the heavy chain CDR1, CDR2 or CDR3 variants or the light chain CDR1, CDR2 or CDR variants comprise at least one amino acid substitution as compared to the parent CDR. In some aspects, the methods of reducing blood glucose levels in a subject further comprise retaining or increasing lean and/or skeletal muscle mass in the subject.

Disclosed herein are methods of reducing pro-inflammatory macrophage buildup in visceral adipose tissue, the methods comprising administering to a subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising the sequence of SEQ ID NO: 19; b) a complementarity determining region 2 (CDR2) comprising the sequence of SEQ ID NO: 20; and/or c) a complementarity determining region (CDR3) comprising the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising the sequence of SEQ ID NO: 49; b) a complementarity determining region 2 (CDR2) comprising the sequence of SEQ ID NO: 50; and/or c) a complementarity determining region 3 (CDR3) comprising the sequence of SEQ ID NO: 51. In some aspects, the methods of reducing pro-inflammatory macrophage buildup in visceral adipose tissue further comprise retaining or increasing lean and/or skeletal muscle mass in the subject.

Disclosed herein are reducing pro-inflammatory macrophage buildup in visceral adipose tissue, the methods comprising administering to a subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising a variant of the sequence of SEQ ID NO: 19; b) a complementarity determining region 2 (CDR2) comprising a variant of the sequence of SEQ ID NO: 20; and/or c) a complementarity determining region (CDR3) comprising a variant of the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising a variant of the sequence of SEQ ID NO: 49; b) a complementarity determining region 2 (CDR2) comprising a variant of the sequence of SEQ ID NO: 50; and/or c) a complementarity determining region 3 (CDR3) comprising a variant of the sequence of SEQ ID NO: 51, wherein any one of the heavy chain CDR1, CDR2 or CDR3 variants or the light chain CDR1, CDR2 or CDR variants comprise at least one amino acid substitution as compared to the parent CDR. In some aspects, the methods of reducing pro-inflammatory macrophage buildup in visceral adipose tissue further comprise retaining or increasing lean and/or skeletal muscle mass in the subject.

Disclosed herein are methods of increasing anti-inflammatory macrophages in visceral adipose tissue, the methods comprising administering to a subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising the sequence of SEQ ID NO: 19; b) a complementarity determining region 2 (CDR2) comprising the sequence of SEQ ID NO: 20; and/or c) a complementarity determining region (CDR3) comprising the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising the sequence of SEQ ID NO: 49; b) a complementarity determining region 2 (CDR2) comprising the sequence of SEQ ID NO: 50; and/or c) a complementarity determining region 3 (CDR3) comprising the sequence of SEQ ID NO: 51. In some aspects, the methods of increasing anti-inflammatory macrophages in visceral adipose tissue further comprise retaining or increasing lean and/or skeletal muscle mass in the subject.

Disclosed herein are methods of increasing anti-inflammatory macrophages in visceral adipose tissue, the methods comprising administering to a subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising a variant of the sequence of SEQ ID NO: 19; b) a complementarity determining region 2 (CDR2) comprising a variant of the sequence of SEQ ID NO: 20; and/or c) a complementarity determining region (CDR3) comprising a variant of the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising a variant of the sequence of SEQ ID NO: 49; b) a complementarity determining region 2 (CDR2) comprising a variant of the sequence of SEQ ID NO: 50; and/or c) a complementarity determining region 3 (CDR3) comprising a variant of the sequence of SEQ ID NO: 51, wherein any one of the heavy chain CDR1, CDR2 or CDR3 variants or the light chain CDR1, CDR2 or CDR variants comprise at least one amino acid substitution as compared to the parent CDR. In some aspects, the methods of increasing anti-inflammatory macrophages in visceral adipose tissue further comprise retaining or increasing lean and/or skeletal muscle mass in the subject.

Disclosed herein are methods of preventing or reducing adipose tissue hypertrophy in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising the sequence of SEQ ID NO: 19; b) a complementarity determining region 2 (CDR2) comprising the sequence of SEQ ID NO: 20; and/or c) a complementarity determining region (CDR3) comprising the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising the sequence of SEQ ID NO: 49; b) a complementarity determining region 2 (CDR2) comprising the sequence of SEQ ID NO: 50; and/or c) a complementarity determining region 3 (CDR3) comprising the sequence of SEQ ID NO: 51. In some aspects, the methods of preventing or reducing adipose tissue hypertrophy in a subject further comprise retaining or increasing lean and/or skeletal muscle mass in the subject.

Disclosed herein are methods of preventing or reducing adipose tissue hypertrophy in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising a variant of the sequence of SEQ ID NO: 19; b) a complementarity determining region 2 (CDR2) comprising a variant of the sequence of SEQ ID NO: 20; and/or c) a complementarity determining region (CDR3) comprising a variant of the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising a variant of the sequence of SEQ ID NO: 49; b) a complementarity determining region 2 (CDR2) comprising a variant of the sequence of SEQ ID NO: 50; and/or c) a complementarity determining region 3 (CDR3) comprising a variant of the sequence of SEQ ID NO: 51, wherein any one of the heavy chain CDR1, CDR2 or CDR3 variants or the light chain CDR1, CDR2 or CDR variants comprise at least one amino acid substitution as compared to the parent CDR. In some aspects, the methods of preventing or reducing adipose tissue hypertrophy in a subject further comprise retaining or increasing lean and/or skeletal muscle mass in the subject.

Disclosed herein are methods of preventing or treating nonalcoholic fatty liver disease or nonalcoholic steatohepatitis in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising the sequence of SEQ ID NO: 19; b) a complementarity determining region 2 (CDR2) comprising the sequence of SEQ ID NO: 20; and/or c) a complementarity determining region (CDR3) comprising the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising the sequence of SEQ ID NO: 49; b) a complementarity determining region 2 (CDR2) comprising the sequence of SEQ ID NO: 50; and/or c) a complementarity determining region 3 (CDR3) comprising the sequence of SEQ ID NO: 51. In some aspects, the methods of preventing or treating nonalcoholic fatty liver disease or nonalcoholic steatohepatitis in a subject further comprise retaining or increasing lean and/or skeletal muscle mass in the subject.

Disclosed herein are methods of preventing or treating nonalcoholic fatty liver disease or nonalcoholic steatohepatitis in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a heavy chain immunoglobulin variable region comprising: a) a complementarity determining region 1 (CDR1) comprising a variant of the sequence of SEQ ID NO: 19; b) a complementarity determining region 2 (CDR2) comprising a variant of the sequence of SEQ ID NO: 20; and/or c) a complementarity determining region (CDR3) comprising a variant of the sequence of SEQ ID NO: 21; and/or wherein the anti-connexin Cx43 hemichannel activating antibody or fragment thereof comprises a light chain immunoglobulin variable region comprising a) a complementarity determining region 1 (CDR1) comprising a variant of the sequence of SEQ ID NO: 49; b) a complementarity determining region 2 (CDR2) comprising a variant of the sequence of SEQ ID NO: 50; and/or c) a complementarity determining region 3 (CDR3) comprising a variant of the sequence of SEQ ID NO: 51, wherein any one of the heavy chain CDR1, CDR2 or CDR3 variants or the light chain CDR1, CDR2 or CDR variants comprise at least one amino acid substitution as compared to the parent CDR. In some aspects, the methods of preventing or treating nonalcoholic fatty liver disease or nonalcoholic steatohepatitis in a subject further comprise retaining or increasing lean and/or skeletal muscle mass in the subject.

In some aspects, any of the methods disclosed herein (e.g., prevent or reduce fat accumulation in a subject, reduce weight gain in a subject, decrease bone marrow adiposity, reduce the volume of visceral and subcutaneous fat in a subject, reduce blood glucose levels, reducing pro-inflammatory macrophage buildup in visceral adipose tissue, increase anti-inflammatory macrophages in visceral adipose tissue, retain or increase lean and/or skeletal muscle mass, or prevent or reduce adipose hypertrophy in a subject; and treating or preventing obesity, diabetes, NASH, or NAFLD) can further comprise administering at least a second therapeutic agent, a second therapy, or a combination thereof to the subject. In some aspects, the second therapeutic agent can be an anti-obesity therapeutic agent or an anti-diabetes therapeutic agent. Examples of anti-obesity therapeutic agents include but are not limited to bupropion-naltrexone (Contrave), liraglutide (Saxenda), Orlistat (Xenical, Alli), phentermine-topiramate (Qsymia), semaglutide (Wegovy), and setmelanotide (Imcivree). Examples of anti-diabetes therapeutic agents include but are not limited to biguanides (metformin), sulfonylureas (DiaBeta, Glynase, Micronase (glyburide or glibenclamide), amaryl (glimepiride), diabinese (chlorpropamide), tolinase (tolazamide), and tolbutamide), meglitinide (repaglinide, nateglinide, and mitiglinide), thiazolidinedione (TZD) (rosiglitazone and pioglitazone), dipeptidyl peptidase 4 (DPP-4) inhibitors (sitagliptin, saxagliptin, linagliptin, and alogliptin), sodium-glucose cotransporter (SGLT2) inhibitors (canagliflozin, dapagliflozin, and empagliflozin), and α-glucosidase inhibitors (acarbose (Precose) and miglitol (Glyset)).

In some aspects, in any of the methods disclosed herein the anti-Cx43 hemichannel activating antibody or fragment thereof can bind to a Cx43 hemichannel. In some aspects, in any of the methods disclosed herein the antibody or fragment thereof can stimulate the opening of a Cx43 hemichannel. In some aspects, in any of the methods disclosed herein the antibody or fragment thereof can stimulate the opening of a Cx43 hemichannel and have no effect on gap junction coupling.

In some aspects, the anti-Cx43 hemichannel activating antibody or fragment thereof can further comprise a tag sequence.

In some aspects, the anti-Cx43 hemichannel activating antibody or fragment thereof can be a Fab fragment an Fab′ fragment or an F(ab′)2 fragment.

Disclosed herein are in vitro methods of using any of the antibodies described herein, compounds or reagents to activate or enhance Cx43 hemichannels. In some aspects, the methods described herein can be used to determine the effect on activation of Cx43 hemichannel opening in osteocytes by (i) determining hemichannel opening by dye uptake assay, using Lucifer yellow or Alexa dyes, (ii) assessing stimulating effects on hemichannels opening by measuring ATP levels (e.g., an increase in ATP release from osteocytes via Cx43 hemichannels can indicate that the compound or antibody being tested can suppressive tumor or cancel cell growth and/or colonization, (iii) test stimulatory effects of the reagents on hemichannels opening by mechanical loading in the form of fluid flow shear stress. In some aspects, the antibody that binds to a connexin 43 (Cx43) hemichannel and enhances channel opening can have no effect on gap channel coupling.

In some aspects, the anti-Cx43 hemichannel activating antibody useful in any of the methods disclosed herein can be an anti-Cx43 hemichannel activating antibody comprising one or more of the sequences set forth in Table 1.

TABLE 1
Examples of sequences of
anti-Cx43 hemichannel activating antibodies.

| SEQ ID | | |
|---|---|---|
| NO: | SEQUENCE | NAME |
| 67 | EVQLVQSGAEVKKPGASVKVSCKASGYTFTSYYMY | Heavy chain |
| | WVRQAPGQGLEWIGGINPSNAGTNFNEKFKNRATL | variable |
| | TVDKSTSTAYMELSSLRSEDTAVYYCTREGNPYYT | domain |
| | MNYWGQGTLVTVSS | |
| 68 | DVVMTQSPLSLPVTIGQPASISCKSSQSLLESDGKTYL | Light chain |
| | NWLQQRPGQSPRRLIYLVSKLDSGVPDRFSGSGSGTD | variable |
| | FTLKISRVEAEDVGVYYCWQGTHFPWTFGGGTKVEIK | domain |
| 69 | EVQLVQSGAEVKKPGASVKVSCKASGYTFTSYYMY | Heavy chain |
| | WVRQAPGQGLEWIGGINPSNGGTNFNEKFKNRATLT | (variable |
| | VDKSTSTAYMELSSLRSEDTAVYYCTREGNPYYTMN | region and |
| | YWGQGTLVTVSSASTKGPSVFPLAPSSKSTSGGTAAL | constant |
| | GCLVKDYFPEPVTVSWNSGALTSGVHTFPAVLQSSGL | region) |
| | YSLSSVVTVPSSSLGTQTYICNVNHKPSNTKVDKKVE | |
| | PKSCDKTHTCPPCPAPELLGGPSVFLFPPKPKDTLMIS | |
| | RTPEVTCVVVDVSHEDPEVKFNWYVDGVEVHNAKT | |
| | KPREEQYNSTYRVVSVLTVLHQDWLNGKEYKCKVS | |
| | NKALPAPIEKTISKAKGQPREPQVYTLPPSRDELTKN | |
| | QVSLTCLVKGFYPSDIAVEWESNGQPENNYKTTPPVL | |
| | DSDGSFFLYSKLTVDKSRWQQGNVFSCSVMHEALHN | |
| | HYTQKSLSLSPGK | |
| 70 | EVQLVQSGAEVKKPGASVKVSCKASGYTFTSYYMY | Heavy chain |
| | WVRQAPGQGLEWIGGINPSNGGTNFNEKFKNRATLT | (variable |
| | VDKSTSTAYMELSSLRSEDTAVYYCTREGNPYYTMN | region and |
| | YVVGQGTLVTVSSASTKGPSVFPLAPCSRSTSESTAAL | constant |
| | GCLVKDYFPEPVTVSWNSGALTSGVHTFPAVLQSSGLY | region) |
| | SLSSVVTVPSSSLGTKTYTCNVDHKPSNTKVDKRVESK | |
| | YGPPCPPCPAPEFLGGPSVFLFPPKPKDTLMISRTPEVTC | |
| | VVVDVSQEDPEVQFNWYVDGVEVHNAKTKPREEQFN | |
| | STYRVVSVLTVLHQDWLNGKEYKCKVSNKGLPSSIEK | |
| | TISKAKGQPREPQVYTLPPSQEEMTKNQVSLTCLVKGF | |
| | YPSDIAVEWESNGQPENNYKTTPPVLDSDGSFFLYSRL | |
| | TVDKSRWQEGNVFSCSVMHEALHNHYTQKSLSLSLGK | |
| 71 | EVQLVQSGAEVKKPGASVKVSCKASGYTFTSYYMYW | Heavy chain |
| | VRQAPGQGLEWIGGINPSNAGTNFNEKFKNRATLTVDK | (variable |
| | STSTAYMELSSLRSEDTAVYYCTREGNPYYTMNYVVG | region and |
| | QGTLVTVSSASTKGPSVFPLAPCSRSTSESTAALGCLVK | constant |
| | DYFPEPVTVSWNSGALTSGVHTFPAVLQSSGLYSLSSV | region) |
| | VTVPSSSLGTKTYTCNVDHKPSNTKVDKRVESKYGPPC | |
| | PPCPAPEFLGGPSVFLFPPKPKDTLMISRTPEVTCVVVDV | |
| | SQEDPEVQFNWYVDGVEVHNAKTKPREEQFNSTYRVV | |
| | SVLTVLHQDWLNGKEYKCKVSNKGLPSSIEKTISKAKG | |
| | QPREPQVYTLPPSQEEMTKNQVSLTCLVKGFYPSDIAVE | |
| | WESNGQPENNYKTTPPVLDSDGSFFLYSRLTVDKSRWQ | |
| | EGNVFSCSVMHEALHNHYTQKSLSLSLGK | |
| 72 | EVQLVQSGAEVKKPGASVKVSCKASGYTFTSYYMYWV | Heavy chain |
| | RQAPGQGLEWIGGINPSNGGTNFNEKFKNRATLTVDKST | (variable |
| | STAYMELSSLRSEDTAVYYCTREGNPYYTMNYWGQGTL | region and |
| | VTVSSASTKGPSVFPLAPSSKSTSGGTAALGCLVKDYFPE | constant |
| | PVTVSWNSGALTSGVHTFPAVLQSSGLYSLSSVVTVPSSS | region) |
| | LGTQTYICNVNHKPSNTKVDKKVEPKSCDKTHTCPPCPA | |
| | PEAAGGPSVFLFPPKPKDTLMISRTPEVTCVVVDVSHEDP | |
| | EVKFNWYVDGVEVHNAKTKPREEQYNSTYRVVSVLTVL | |
| | HQDWLNGKEYKCKVSNKALPAPIEKTISKAKGQPREPQV | |
| | YTLPPSRDELTKNQVSLTCLVKGFYPSDIAVEWESNGQP | |
| | ENNYKTTPPVLDSDGSFFLYSKLTVDKSRWQQGNVFSCS | |
| | VMHEALHNHYTQKSLSLSPGK | |
| 73 | EVQLVQSGAEVKKPGASVKVSCKASGYTFTSYYMYWV | Heavy chain |
| | RQAPGQGLEWIGGINPSNAGTNFNEKFKNRATLTVDKST | (variable |
| | STAYMELSSLRSEDTAVYYCTREGNPYYTMNYWGQGT | region and |
| | LVTVSSASTKGPSVFPLAPSSKSTSGGTAALGCLVKDYF | constant |
| | PEPVTVSWNSGALTSGVHTFPAVLQSSGLYSLSSVVTVP | region) |
| | SSSLGTQTYICNVNHKPSNTKVDKKVEPKSCDKTHTCPP | |
| | CPAPEAAGGPSVFLFPPKPKDTLMISRTPEVTCVVVDVS | |
| | HEDPEVKFNWYVDGVEVHNAKTKPREEQYNSTYRVVS | |
| | VLTVLHQDWLNGKEYKCKVSNKALPAPIEKTISKAKGQ | |
| | PREPQVYTLPPSRDELTKNQVSLTCLVKGFYPSDIAVEW | |
| | ESNGQPENNYKTTPPVLDSDGSFFLYSKLTVDKSRWQQ | |
| | GNVFSCSVMHEALHNHYTQKSLSLSPGK | |
| 74 | EVQLVQSGAEVKKPGASVKVSCKASGYTFTSYYMYWV | Heavy chain |
| | RQAPGQGLEWIGGINPSNGGTNFNEKFKNRATLTVDKST | (variable |
| | STAYMELSSLRSEDTAVYYCTREGNPYYTMNYWGQGTL | region and |
| | VTVSSASTKGPSVFPLAPCSRSTSESTAALGCLVKDYFPE | constant |
| | PVTVSWNSGALTSGVHTFPAVLQSSGLYSLSSVVTVPSSS | region) |
| | LGTKTYTCNVDHKPSNTKVDKRVESKYGPPCPPCPAPEF | |
| | EGGPSVFLFPPKPKDTLMISRTPEVTCVVVDVSQEDPEVQ | |
| | FNWYVDGVEVHNAKTKPREEQFNSTYRVVSVLTVLHQD | |
| | WLNGKEYKCKVSNKGLPSSIEKTISKAKGQPREPQVYTL | |
| | PPSQEEMTKNQVSLTCLVKGFYPSDIAVEWESNGQPENN | |
| | YKTTPPVLDSDGSFFLYSRLTVDKSRWQEGNVFSCSVMH | |
| | EALHNHYTQKSLSLSLGK | |
| 75 | EVQLVQSGAEVKKPGASVKVSCKASGYTFTSYYMYWVR | Heavy chain |
| | QAPGQGLEWIGGINPSNAGTNFNEKFKNRATLTVDKSTST | (variable |
| | AYMELSSLRSEDTAVYYCTREGNPYYTMNYWGQGTLVT | region and |
| | VSSASTKGPSVFPLAPCSRSTSESTAALGCLVKDYFPEPVT | constant |
| | VSWNSGALTSGVHTFPAVLQSSGLYSLSSVVTVPSSSLGT | region) |
| | KTYTCNVDHKPSNTKVDKRVESKYGPPCPPCPAPEFEGG | |
| | PSVFLFPPKPKDTLMISRTPEVTCVVVDVSQEDPEVQFNW | |
| | YVDGVEVHNAKTKPREEQFNSTYRVVSVLTVLHQDWLN | |
| | GKEYKCKVSNKGLPSSIEKTISKAKGQPREPQVYTLPPSQ | |
| | EEMTKNQVSLTCLVKGFYPSDIAVEWESNGQPENNYKTT | |
| | PPVLDSDGSFFLYSRLTVDKSRWQEGNVFSCSVMHEALH | |
| | NHYTQKSLSLSLGK | |
| 76 | EVQLVQSGAEVKKPGASVKVSCKASGYTFTSYYMYWVR | Heavy chain |
| | QAPGQGLEWIGGINPSNGGTNFNEKFKNRATLTVDKSTST | (variable |
| | AYMELSSLRSEDTAVYYCTREGNPYYTMNYWGQGTLVT | region and |
| | VSSASTKGPSVFPLAPCSRSTSESTAALGCLVKDYFPEPVT | constant |
| | VSWNSGALTSGVHTFPAVLQSSGLYSLSSVVTVPSSSLGT | region) |
| | KTYTCNVDHKPSNTKVDKRVESKYGPPCPPCPAPEAAGG | |
| | PSVFLFPPKPKDTLMISRTPEVTCVVVDVSQEDPEVQFNW | |
| | YVDGVEVHNAKTKPREEQFNSTYRVVSVLTVLHQDWLN | |
| | GKEYKCKVSNKGLPSSIEKTISKAKGQPREPQVYTLPPSQ | |
| | EEMTKNQVSLTCLVKGFYPSDIAVEWESNGQPENNYKTT | |
| | PPVLDSDGSFFLYSRLTVDKSRWQEGNVFSCSVMHEALH | |
| | NHYTQKSLSLSLGK | |
| 77 | EVQLVQSGAEVKKPGASVKVSCKASGYTFTSYYMYWVR | Heavy chain |
| | QAPGQGLEWIGGINPSNAGTNFNEKFKNRATLTVDKSTST | (variable |
| | AYMELSSLRSEDTAVYYCTREGNPYYTMNYWGQGTLVT | region and |
| | VSSASTKGPSVFPLAPCSRSTSESTAALGCLVKDYFPEPVT | constant |
| | VSWNSGALTSGVHTFPAVLQSSGLYSLSSVVTVPSSSLGT | region) |
| | KTYTCNVDHKPSNTKVDKRVESKYGPPCPPCPAPEAAGG | |
| | PSVFLFPPKPKDTLMISRTPEVTCVVVDVSQEDPEVQFNW | |
| | YVDGVEVHNAKTKPREEQFNSTYRVVSVLTVLHQDWL | |
| | NGKEYKCKVSNKGLPSSIEKTISKAKGQPREPQVYTLPP | |
| | SQEEMTKNQVSLTCLVKGFYPSDIAVEWESNGQPENNY | |
| | KTTPPVLDSDGSFFLYSRLTVDKSRWQEGNVFSCSVMH | |
| | EALHNHYTQKSLSLSLGK | |
| 78 | DVVMTQSPLSLPVTIGQPASISCKSSQSLLESDGKTYLNW | Light chain |
| | LQQRPGQSPRRLIYLVSKLDSGVPDRFSGSGSGTDFTLKI | (variable |
| | SRVEAEDVGVYYCWQGTHFPWTFGGGTKVEIKRTVAA | region and |
| | PSVFIFPPSDEQLKSGTASVVCLLNNFYPREAKVQWKVD | constant |
| | NALQSGNSQESVTEQDSKDSTYSLSSTLTLSKADYEKHK | region) |
| | VYACEVTHQGLSSPVTKSFNRGEC | |

### Treatment of Diseases

Disclosed herein are anti-Cx43 hemichannel activating antibodies (e.g., the M2 antibody) and biological fragments thereof that can be used to prevent or reduce fat accumulation in a subject, reduce weight gain in a subject, decrease bone marrow adiposity, reduce the volume of visceral and subcutaneous fat in a subject, retain or increase lean and/or skeletal muscle mass reduce blood glucose levels, reducing pro-inflammatory macrophage buildup in visceral adipose tissue, increase anti-inflammatory macrophages in visceral adipose tissue, or prevent or reduce adipose hypertrophy in a subject; and treating or preventing obesity, diabetes (type II), NASH, or NAFLD in a subject in need thereof with antibodies that activate Cx43 hemichannel opening.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with, diagnosed or at risk for obesity, diabetes (type II), NASH, or NAFLD in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the disease or condition is delayed, hindered, or prevented, or the disease or condition or a symptom of the disease or condition is ameliorated or its frequency can be reduced. One or more of the symptoms can be less severe. Recovery can be accelerated in an individual who has been treated. For example, treatment of obesity, type II diabetes, NASH or NAFLD may involve, for example, preventing or reducing fat accumulation, reducing weight gain, decreasing bone marrow adiposity, reducing the volume of visceral and subcutaneous fat, reducing blood glucose levels, reducing pro-inflammatory macrophage buildup in visceral adipose tissue, increasing anti-inflammatory macrophages in visceral adipose tissue, retaining or increasing lean and/or skeletal muscle mass, or preventing or reducing adipose tissue hypertrophy. In some aspects, the anti-Cx43 hemichannel activating antibodies described herein can improve the quality of life of a subject with or at risk for obesity, diabetes (type II), NASH, or NAFLD. In some aspects, the anti-Cx43 hemichannel activating antibodies described herein can prevent or reduce fat accumulation in a subject, reduce weight gain in a subject, decrease bone marrow adiposity, reduce the volume of visceral and subcutaneous fat in a subject, retain or increase lean and/or skeletal muscle mass, reduce blood glucose levels, reducing pro-inflammatory macrophage buildup in visceral adipose tissue, increase anti-inflammatory macrophages in visceral adipose tissue, or prevent or reduce adipose hypertrophy in a subject; and treat or prevent obesity, diabetes, NASH, or NAFLD.

In some aspects, the methods can comprise administering an effective amount of the anti-Cx43 hemichannel activating antibody to the subject. In some aspects, the method can comprise administering an effective amount of an expression vector encoding the anti-Cx43 hemichannel activating antibody to the subject. In some aspects, the anti-Cx43 hemichannel activating antibody that binds to a connexin 43 (Cx43) hemichannel and enhances channel opening can have no effect on gap channel coupling.

In some aspects, the subject, prior to administration of an effective amount of the anti-Cx43 hemichannel activating antibody, has been diagnosed with obesity, diabetes (type II), NASH, or NAFLD prior to the administering step.

The compositions described herein can be formulated to include a therapeutically effective amount of the anti-Cx43 hemichannel activating antibodies disclosed herein. In some aspects, antibodies disclosed herein can be contained within a pharmaceutical formulation. In some aspects, the pharmaceutical formulation can be a unit dosage formulation.

The therapeutically effective amount or dosage of any of the antibodies used in the methods as disclosed herein applied to mammals (e.g., humans) can be determined by one of ordinary skill in the art with consideration of individual differences in age, weight, sex, the severity of the subject's symptoms, and the particular composition or route of administration selected, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the obesity, diabetes, NASH, or NAFLD symptoms), the age and physical characteristics of the subject and other considerations known to those of ordinary skill in the art. Dosages can be established using clinical approaches known to one of ordinary skill in the art. A therapeutically effective dosage of an anti-hemichannel antibody (e.g., anti-Cx43 hemichannel activating antibody) can result in a decrease in severity of one or more disease symptoms, an increase in frequency and duration of disease symptom-free periods, or a prevention of impairment or disability due to the disease affliction. A therapeutically effective amount of a therapeutic compound or anti-Cx43 hemichannel activating antibody can prevent or reduce fat accumulation in a subject, reduce weight gain in a subject, retain or increase lean and/or skeletal muscle mass, decrease bone marrow adiposity, reduce the volume of visceral and subcutaneous fat in a subject, reduce blood glucose levels, reducing pro-inflammatory macrophage buildup in visceral adipose tissue, increase anti-inflammatory macrophages in visceral adipose tissue, or prevent or reduce adipose hypertrophy, or otherwise ameliorate symptoms in a subject.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The total effective amount of the anti-Cx43 hemichannel activating antibodies or compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The anti-Cx43 hemichannel activating antibodies or compositions described herein can be administered in conjunction with other therapeutic modalities to a subject in need of therapy. The present compounds can be given to prior to, simultaneously with or after treatment with other agents or regimes. For example, the anti-Cx43 hemichannel activating antibodies disclosed herein can be administered alone or in conjunction with standard therapies used to obesity, diabetes, NASH, or NAFLD. In some aspects, any of the anti-Cx43 hemichannel activating antibody or compositions described herein can be administered or used together with therapeutic agents used to treat obesity or type II diabetes.

### Pharmaceutical Compositions

Disclosed herein are compositions, e.g., pharmaceutical compositions, comprising one or a combination of the anti-Cx43 hemichannel activating antibodies, or antigen-binding portion(s) thereof formulated with a pharmaceutically acceptable carrier. Such compositions may include one or a combination of (e.g., two or more different) anti-Cx43 hemichannel activating antibodies, or immunoconjugates described herein. For example, a pharmaceutical composition of the invention can comprise a combination of anti-Cx43 hemichannel activating antibodies that bind to different epitopes on the target antigen or that have complementary activities.

Pharmaceutical compositions of the invention also can be administered as combination therapy, i.e., combined with other agents. For example, the combination therapy can include an anti-Cx43 hemichannel activating antibody combined with at least one other therapeutic agent or therapy. In some aspects, the second therapeutic agent can be an anti-obesity therapeutic agent or an anti-diabetes therapeutic agent.

As used herein, the phrase “pharmaceutically acceptable carrier” includes any solvents, dispersion media, coatings, antibacterial and antifungal agents, isotonic and absorption delaying agents, and the like that are physiologically compatible. Preferably, the carrier can be suitable for intravenous, intramuscular, subcutaneous, or parenteral administration (e.g., by injection or infusion). Depending on the route of administration, the active compound, i.e., antibody, or immunoconjugate, may be coated in a material to protect the compound from the action of acids and other natural conditions that may inactivate the compound.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated herein, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention is dictated by and directly dependent on (a) the unique characteristics of the active compound and the particular therapeutic effect to be achieved, and (b) the limitations inherent in the art of compounding such an active compound for the treatment of sensitivity in individuals.

For administration of the anti-Cx43 hemichannel activating antibody, the dosage ranges from about 0.0001 to 100 mg/kg, and more usually 0.01 to 5 mg/kg, 5 mg/kg to 10 mg/kg, 10 mg/kg to 15 mg/kg, 15 mg/kg to 20 mg/kg or 20 mg/kg to 25 mg/kg of the host body weight. In some aspects, the dosages can be 0.3 mg/kg body weight, 1 mg/kg body weight, 3 mg/kg body weight, 5 mg/kg body weight or 10 mg/kg body weight or within the range of 1-10 mg/kg. In some aspects, the dosages can be 0.3 mg/kg body weight, 1 mg/kg body weight, 3 mg/kg body weight, 5 mg/kg body weight, 10 mg/kg body weight, 15 mg/kg body weight, 20 mg/kg body weight, 25 mg/kg body weight or 30 mg/kg body weight or within the range of 1-30 mg/kg. In some aspects, the dosages can be about 15, 16, 17, 18, 19, 20, 21, 22, 23, 24 or 25 mg/kg body weight. In some aspects, the dosages can be 5 mg/kg body weight. In some aspects, the dosages can be 15 mg/kg body weight. In some aspects, the dosages can be 20 mg/kg body weight. In some aspects, the dosages can be 25 mg/kg body weight. An exemplary treatment regime entails administration once per week, once every two weeks, once every three weeks, once every four weeks, once a month, once every 3 months or once every three to 6 months. Preferred dosage regimens for the anti-Cx43 hemichannel activating antibody of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

In some methods, the anti-Cx43 hemichannel activating antibodies or fragments disclosed herein with different binding specificities can be administered simultaneously, in which case the dosage of each anti-Cx43 hemichannel activating antibody administered falls within the ranges indicated. The anti-Cx43 hemichannel activating antibody is usually administered on multiple occasions. Intervals between single dosages can be, for example, weekly, monthly, every three months or yearly. Intervals can also be irregular as indicated by measuring blood levels of the anti-Cx43 hemichannel activating antibody to the target antigen in the patient. In some methods, dosage is adjusted to achieve a plasma anti-Cx43 hemichannel activating antibody concentration of about 1-1000 μg/ml and in some methods about 25-300 μg/ml.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A composition of the present invention can be administered via one or more routes of administration using one or more of a variety of methods known in the art. As will be appreciated by the skilled artisan, the route and/or mode of administration will vary depending upon the desired results. Preferred routes of administration for antibodies of the invention include intravenous, intramuscular, intradermal, intraperitoneal, subcutaneous, or other parenteral routes of administration, for example by injection or infusion. The phrase “parenteral administration” as used herein means modes of administration other than enteral and topical administration, usually by injection, and includes, without limitation, intravenous, intramuscular, intraarterial, intraperitoneal, transtracheal, subcutaneous, subcuticular, intraarticular injection and infusion.

In some aspects, the anti-Cx43 hemichannel activating antibody disclosed herein can be administered systemically. In some aspects, the antibody disclosed herein can be administered intravenously, intradermally, intratumorally, intramuscularly, intraperitoneally, subcutaneously, or locally.

Combination Treatments. The compositions and methods described herein can involve an anti-Cx43 hemichannel activating antibody or an anti-Cx43 hemichannel activating antibody fragment thereof against Cx43 to stimulate the opening of the Cx43 hemichannel to, for example, prevent or reduce fat accumulation in a subject, reduce weight gain in a subject, decrease bone marrow adiposity, reduce the volume of visceral and subcutaneous fat in a subject, reduce blood glucose levels, reduce pro-inflammatory macrophage buildup in visceral adipose tissue, increase anti-inflammatory macrophages in visceral adipose tissue, or prevent or reduce adipose hypertrophy in a subject; and treat or prevent obesity, diabetes, NASH, or NAFLD, in combination with a second therapeutic or additional therapy.

The methods and compositions, including combination therapies, enhance the therapeutic or protective effect, and/or increase the therapeutic effect of another therapeutic or therapy. Therapeutic and prophylactic methods and compositions can be provided in a combined amount effective to achieve the desired effect. This process may involve contacting the cells with both an anti-Cx43 hemichannel activating antibody or anti-Cx43 hemichannel activating antibody fragment and a second therapy. A tissue or cell can be contacted with one or more compositions or pharmacological formulation(s) comprising one or more of the agents (e.g., anti-Cx43 hemichannel activating antibody or anti-Cx43 hemichannel activating antibody fragment or an anti-obesity therapeutic agent or an anti-diabetes therapeutic agent), or by contacting the tissue and/or cell with two or more distinct compositions or formulations, wherein one composition provides 1) an anti-Cx43 hemichannel activating antibody or anti-Cx43 hemichannel activating antibody fragment, 2) an anti-obesity therapeutic agent or an anti-diabetes therapeutic agent, or 3) both an anti-Cx43 hemichannel activating antibody or anti-Cx43 hemichannel activating antibody fragment and an anti-obesity therapeutic agent or an anti-diabetes therapeutic agent.

The terms “contacted” and “exposed,” when applied to a cell, are used herein to describe the process by which a therapeutic agent is delivered to a target cell or are placed in direct juxtaposition with the target cell.

The anti-Cx43 hemichannel activating antibodies and biological fragments thereof can be administered before, during, after, or in various combinations relative to any second treatment or therapy. The administrations may be in intervals ranging from concurrently to minutes to days to weeks. In aspects where the anti-Cx43 hemichannel activating antibody or anti-Cx43 hemichannel activating antibody fragment is provided to a patient separately from an anti-obesity therapeutic agent or an anti-diabetes therapeutic agent, one would generally ensure that a significant period of time did not expire between the time of each delivery, such that the two compounds would still be able to exert an advantageously combined effect on the patient. In such instances, it is contemplated that one may provide a patient with the anti-Cx43 hemichannel activating antibody therapy and an anti-obesity therapeutic agent or an anti-diabetes therapeutic agent within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In some aspects, a course of treatment can last between 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there can be a period of time at which no second treatment or therapy is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below an antibody therapy is “A” and a second therapy is “B”:
A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A.

Administration of any compound or therapy disclosed herein to a patient will follow general protocols for the administration of such compounds, taking into account the toxicity, if any, of the agents. Therefore, in some aspects there can be a step of monitoring toxicity that can be attributable to combination therapy.

### Kits and Diagnostics

Disclosed herein are kits comprising one or more therapeutic agents and/or other therapeutic and delivery agents. In some aspects, the kit can be used for preparing and/or administering a therapy disclosed herein. The kit may comprise one or more sealed vials containing any of the compositions disclosed herein. The kit may include, for example, at least one anti-Cx43 hemichannel activating antibody or fragment thereof as well as reagents to prepare, formulate, and/or administer the components one or more of the compositions disclosed herein or perform one or more steps of the inventive methods. In some aspects, the kit may also comprise a suitable container, which can be a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### EXAMPLES

It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Anti-Cx43 Monoclonal Antibodies

Anti-Cx43 monoclonal antibodies were generated and clones were identified that produced anti-Cx43-binding monoclonal antibodies. CDR sequences of both DNA and amino acids for the antibody sequences are shown in the tables below along with the correct pairing for each of the characterized antibodies. The M1 antibody inhibits the opening of a Cx43 hemichannel. The M2 antibody activates, stimulates and/or enhances the opening of a Cx43 hemichannel.

TABLE 2
Pairing of heavy chain and light chain
for two functional antibodies.

| Antibody Name | Heavy chain | Light chain |
|---|---|---|
| M1 (HmAb1) | M1H | M1K1 |
| M2 (HmAb2) | M1H | M1M7K |

TABLE 3
Sequence of antibody chains from the hybridomas.

| mAb | CDR-1 | CDR-2 | CDR-3 |
|---|---|---|---|
| M1H | ggctacaccttcacc | attaatcctagcaat | acaagagagggtaac |
| | agctactat | ggtggtact | ccctactatactatg |
| | (SEQ ID NO: 16) | (SEQ ID NO: 17 | aactac |
| | | | (SEQ ID NO: 18) |
| | GYTFTSYY | INPSNGGT | TREGNPYYTMNY |
| | (SEQ ID NO: 19) | (SEQ ID NO: 20) | (SEQ ID NO: 21) |
| M7H | ggctacatcttcacc | attagtcctagcaac | gcacgattcgacgag |
| | acctactgg | ggtcgttct | ggggacttc |
| | (SEQ ID NO: 22) | (SEQ ID NO: 23) | (SEQ ID NO: 24) |
| | GYIFITYW | ISPSNGRS | ARFDEGDF |
| | (SEQ ID NO: 25) | (SEQ ID NO: 26) | (SEQ ID NO: 27) |
| M7Ha | GYIFITTW | ISPSNGRS | ARFDEGDF |
| | (SEQ ID NO: 64) | (SEQ ID NO: 26) | (SEQ ID NO: 27) |
| M1K1 | cagagtctgttaaac | ggggcatcc | cagaatgatcatagt |
| | agtggaaatcaaaag | (SEQ ID NO: 29) | tatccattcacg |
| | acctac | | (SEQ ID NO: 30) |
| | (SEQ ID NO: 28) | | |
| | QSLLNSGNQKTY | GAS | QNDYSYPFT |
| | (SEQ ID NO: 31) | (SEQ ID NO: 32) | (SEQ ID NO: 33) |
| M1K1a | QSLLNSGNQKTY | GAS | QNDHSYPFT |
| | (SEQ ID NO: 31) | (SEQ ID NO: 32) | (SEQ ID NO: 65) |
| M1K2 | aaaagtgtcagtaca | cttgtatcc | cagcacattagggag |
| | tctggctatagttat | (SEQ ID NO: 35) | cttacacg |
| | (SEQ ID NO: 34) | | (SEQ ID NO: 36) |
| | KSVSTSGYSY | LVS | QHIRELT |
| | (SEQ ID NO: 37) | (SEQ ID NO: 38) | (SEQ ID NO: 39) |
| M2K | aaaagtgtcagtaca | cttgtatcc | cagcacattagggag |
| | tctggctatagttat | (SEQ ID NO: 41) | cttacacgt |
| | (SEQ ID NO: 40) | | (SEQ ID NO: 42) |
| | KSVSTSGYSY | LVS | QHIRELTR |
| | (SEQ ID NO: 43) | (SEQ ID NO: 44) | (SEQ ID NO: 45) |
| M1M7K | gagcctcttagaaag | ctggtgtct | tggcaaggtacacat |
| | cgatggaaagacata | (SEQ ID NO: 47) | tttccgtggacg |
| | t | | (SEQ ID NO: 48) |
| | (SEQ ID NO: 46) | | |
| | QSLLESDGKTY | LVS | WQGTHFPWT |
| | (SEQ ID NO: 49) | (SEQ ID NO: 50) | (SEQ ID NO: 51) |

Cloned variable domains are shown in the charts below.

TABLE 4
DNA sequences.
Variable heavy chain (bold)
and variable light chain (underlined).

M1H:
GAGGTCCAACTCCAGCAGCCTGGGGCTGAACTGGTGAAGCCTGGGG
CTTCAGTGAAGTTGTCCTGCAAGGCTTCTGGCTACACCTTCACCAG
CTACTATATGTACTGGGTGAAGCAGAGGCCTGGACAAGGCCTTGAG
TGGATTGGGGGAATTAATCCTAGCAATGGTGGTACTAACTTCAATG
AGAAGTTCAAGAACAAGGCCACACTGACTGTAGACAAATCCTCCAG
CACAGCCTACATGCAACTCAGCAGCCTGACATCTGAGGACTCTGCG
GTCTATTACTGTACAAGAGAGGGTAACCCCTACTATACTATGAACT
ACTGGGGTCAAGGAACCTCAGTCACCGTCTCCTCA (SEQ ID
NO: 52)
M7H:
GAGGTCCAACTCCAGCAACCTGGGGCTGAACTGGTGAGGCCTGGGG
CTTCAGTAATGCTGTCCTGCAAGGCTTCTGGCTACATCTTCACCAC
CTACTGGATGCACTGGCTGAAGCAGAGGCCTGGACAAGGCCTTGAC
TGGATTGGAGAGATTAGTCCTAGCAACGGTCGTTCTAATTACAATA
AGAAGTTCAAGAGCAAGGCCACACTGACTGTAGACAAATCCTCCAG
CACAGCCTACATGCAACTCAGCAGCCTGACATCTGAGGACTCTGCG
GTCTATTACTGTGCACGATTCGACGAGGGGGACTTCTGGGGCCAAG
GCACCACTCTCATAGTCTCCTCA (SEQ ID NO: 53)
M1K1:
GACATTGTGATGACGCAGTCTCCATCCTCCCTGAGTGTGTCAGCAG
GAGAGAAGGTCACTATGAGCTGCAAGTCCAGTCAGAGTCTGTTAAA
CAGTGGAAATCAAAAGACCTACTTGGCCTGGTACCAGCAGAAACCA
GGGCAGCCTCCTAAACTGTTGATCTACGGGGCATCCACTAGGGAAT
CTGGGGTCCCTGATCGCTTCACAGGCAGTGGATCTGGAACCGATTT
CACTCTTACCATCAGCAGTGTGCAGGCTGAAGACCTGGCAGTTTAT
TACTGTCAGAATGATCATAGTTATCCATTCACGTTCGGCTCGGGGA
CAAAGTTGGAAATAAAA (SEQ ID NO: 54)
M1K2:
GACATTGTGTTGACACAGTCTCCTGCTTCCTTAGCTGTATCTCTGG
GGCAGAGGGCCACCATCTCATACAGGGCCAGCAAAAGTGTCAGTAC
ATCTGGCTATAGTTATATGCACTGGAACCAACAGAAACCAGGACAG
CCACCCAGACTCCTCATCTATCTTGTATCCAACCTAGAATCTGGGG
TCCCTGCCAGGTTCAGTGGCAGTGGGTCTGGGACAGACTTCACCCT
CAACATCCATCCTGTGGAGGAGGAGGATGCTGCAACCTATTACTGT
CAGCACATTAGGGAGCTTACACGTTCGGAGGGGGGACCAAGCTGGA
AATCAAAC (SEQ ID NO: 55)
M2K:
GATATTGTGATGACCCAGTCTCCCGCTTCCTTAGCTGTATCTCTGG
GGCAGAGGGCCACCATCTCATACAGGGCCAGCAAAAGTGTCAGTAC
ATCTGGCTATAGTTATATGCACTGGAACCAACAGAAACCAGGACAG
CCACCCAGACTCCTCATCTATCTTGTATCCAACCTAGAATCTGGGG
TCCCTGCCAGGTTCAGTGGCAGTGGGTCTGGGACAGACTTCACCCT
CAACATCCATCCTGTGGAGGAGGAGGATGCTGCAACCTATTACTGT
CAGCACATTAGGGAGCTTACACGTTCGGAGGGGGGGACCAAGCTGG
AAATCAAA (SEQ ID NO: 56)
M1M7K:
GACGTTGTGATGACCCAGACTCCACTCACTTTGTCGGTTACCATTG
GACAACCAGCCTCCATCTCTTGCAAGTCAAGTCAGAGCCTCTTAGA
AAGCGATGGAAAGACATATTTGAATTGGTTGTTACAGAGGCCAGGC
CAGTCTCCAAAGCGCCTAATCTATCTGGTGTCTAAACTGGACTCTG
GAGTCCCTGACAGGTTCACTGGCAGTGGATCAGGGACAGATTTCAC
ACTGAAAATCAGCAGAGTGGAGGCTGAGGATTTGGGAGTTTATTAT
TGCTGGCAAGGTACACATTTTCCGTGGACGTTCGGTGGAGGCACCA
AGCTGGAAATCAAA (SEQ ID NO: 57)

TABLE 5
Amino acid sequences.
Variable heavy chain (bold)
and variable light chain (underlined).

M1H:
EVQLQQPGAELVKPGASVKLSCKASGYTFTSYYMYWVKQRPGQG
LEWIGGINPSNGGTNFNEKFKNKATLTVDKSSSTAYMQLSSLTS
EDSAVYYCTREGNPYYTMNYWGQ GTSVTVSS (SEQ ID NO:
58)
M7H:
EVQLQQPGAELVRPGASVMLSCKASGYIFTTYWMHWLKQRPGQG
LDWIGEISPSNGRSNYNKKFKSKATLTVDKSSSTAYMQLSSLTS
EDSAVYYCARFDEGDFWGQGTTLIVS (SEQ ID NO: 59)
M1K1:
DIVMTQSPSSLSVSAGEKVTMSCKSSQSLLNSGNQKTYLAWYQQ
KPGQPPKLLIYGASTRSGVPDRFTGSGSGTDFTLTISSVQAEDL
AVYYCQNDHSYPFTFGSGTKLEIK (SEQ ID NO: 60)
M1K2:
DIVLTQSPASLAVSLGQRATISYRASKSVSTSGYSYMHWNQQKP
GQPPRLLIYLVSNLESGVP ARFSGSGSGTDFTLNIHPVEEEDA
ATYYCQHIRELTRSEGGPSWKSN (SEQ ID NO: 61)
M2-K:
DIVMTQSPASLAVSLGQRATISYRASKSVSTSGYSYMHWNQQKP
GQPPRLLIYLVSNLESGVPARFSGSGSGTDFTLNIHPVEEEDAA
TYYCQHIRELTRSEGGTKLEIK (SEQ ID NO: 62)
M1M7-K:
DVVMTQTPLTLSVTIGQPASISCKSSQSLLESDGKTYLNWLLQR
PGQSPKRLIYLVSKLDSGVPDRFTGSGSGTDFTLKISRVEAEDL
GVYYCWQGTHFPWTFGGGTKLEIK (SEQ ID NO: 63)

### Example 2—Anti-Connexin Cx43 Hemichannel Activating Antibody Opens Cx43 Hemichannels

MLO-Y4 osteocytes and primary mouse osteocytes were incubated with E2 (polyclonal), a humanized anti-connexin Cx43 hemichannel activating antibody (M2 antibody; HmAb2), and a humanized anti-connexin Cx43 hemichannel blocking antibody (M1 antibody; HmAb1) or carbenoxolone (CBX), a connexin channel blocker. Ethidium bromide (EtBr) dye uptake assay was performed. The results shown in FIGS. 10A-B demonstrate that the humanized anti-connexin Cx43 hemichannel activating antibody (M2 antibody; HmAb2) actives Cx43 hemichannels.

### Example 3—Osteocytic Connexin Hemichannel Regulation of Extramedullary Adiposity

Obesity is a global epidemic and major contributor to the leading causes of death in the U.S., including diabetes, heart disease, stroke, and certain types of cancer. Maintenance of whole-body metabolic homeostasis involves the coordination of multiple tissues, including adipose tissue and bone. Given its prominent role in multiple bodily processes, recent evidence points to bone as a significant player in whole-body energy metabolism. Osteocytes comprise >90% of bone cells, are mechanosensors, and orchestrators of the bone remodeling process. Osteocytic connexin 43 (Ocy Cx43) is a transmembrane protein expressed in bone that forms hemichannels (HCs) that facilitate the communication of cells among themselves and with their environment. The role of Ocy Cx43 in the modulation of adipose tissue, metabolism, and disease progression, is described herein.

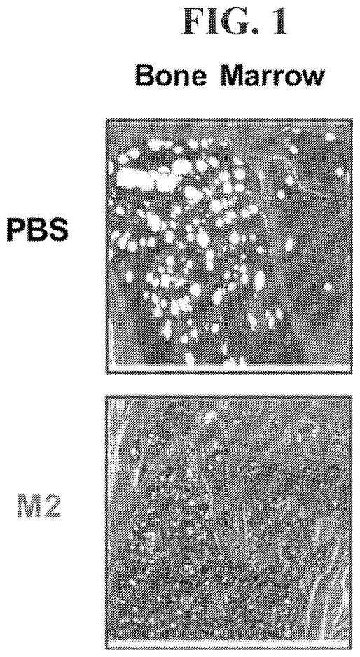

Using a monoclonal anti-Cx43 hemichannel activating antibody (identified as the M2 antibody herein) that specifically opens Cx43 hemichannels plus exposure to 60% Kcal high fat diet (HFD), the results demonstrate that the opening of Ocy Cx43 hemichannels using the M2 antibody reduces total adipose tissue (both medullary and extramedullary, measured using adipose tissue weights and Dual-energy X-ray absorptiometry (DEXA)), and improves metabolic parameters, such as % Fat, glucose, and M1/M2 macrophage ratios (see, FIGS. 1-9).

Methods. Experimental animals. 16-week-old WT male mice fed a 60% Kcal HFD for 10 weeks, were bought (B6 DIO, JAX); post-acclimation, mice remained in 60% HFD conditions and underwent ±M2 antibody (25 mg/kg) treatment for 4 weeks (1×/per week, IP injection).

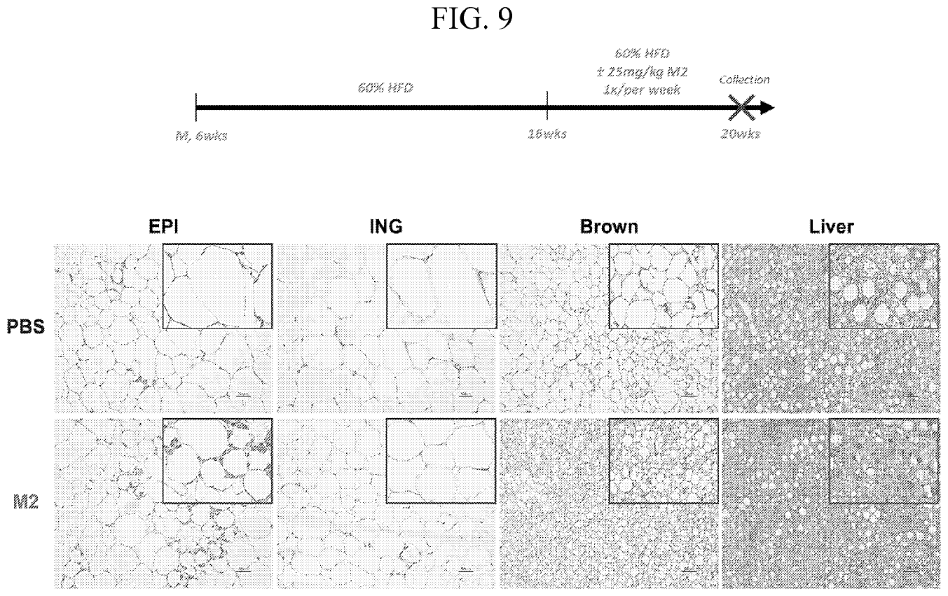

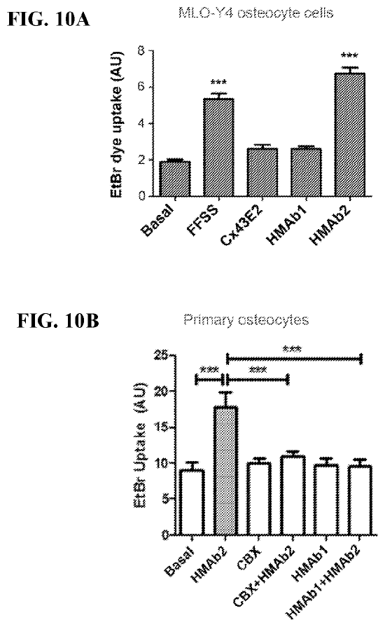

Histology. Adipose tissue, liver, and tibia bone samples were isolated from experimental animals and fixed with 4% formaldehyde. Bone samples underwent decalcification. The samples were then processed, paraffin-embedded, sliced, and stained with hematoxylin & eosin (H&E) to visualize adipocytes in the bone marrow and epididymal, inguinal, brown, and liver tissue. The results are shown in FIGS. 1 and 9.

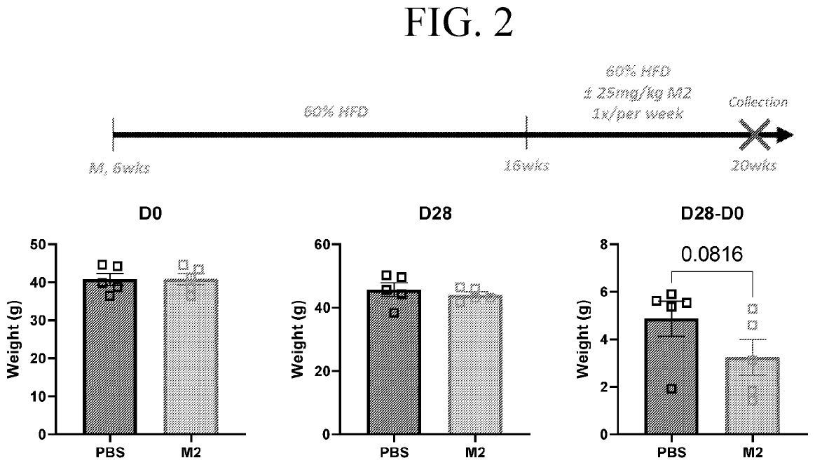

Mice and tissue weights. Throughout the study, mice were weighed weekly, and weight fluctuations were calculated post- and pre-treatment in the ±M2 antibody groups. At the time of sacrifice, isolated adipose tissue samples were collected and weighed. This tissue weight was then normalized to the corresponding mice weight that day to obtain “percent to weight”. Brown:white fat ratio=brown (%)/(EPI (%)+ING (%). Visceral:subcutaneous fat ratio=EPI (%)/ING (%). The results are shown in FIGS. 2, 4, and 5.

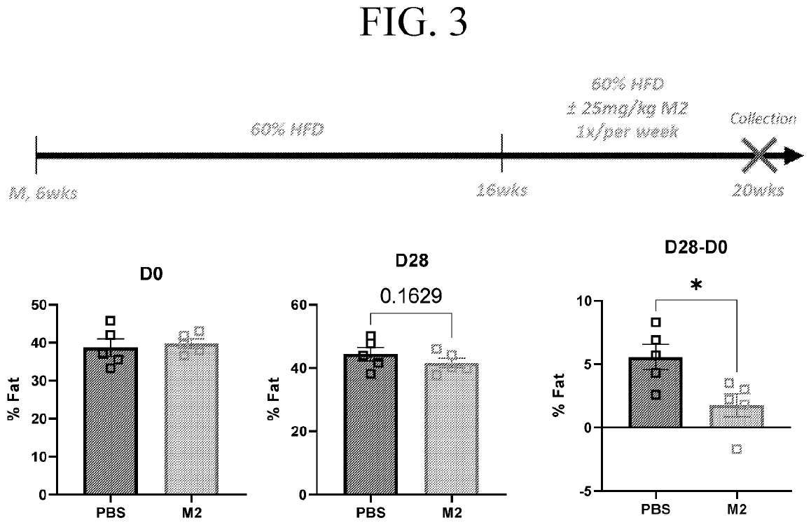

Dual-energy X-ray absorptiometry (DEXA) fat % analysis. Mice were anesthetized, and body composition was determined with a PIXImus2 Mouse Densitometer (GE Medical Systems, Madison, WI) on day 0 and day 28 of the experiment to determine % fat changes M2 antibody. The results are shown in FIG. 3.

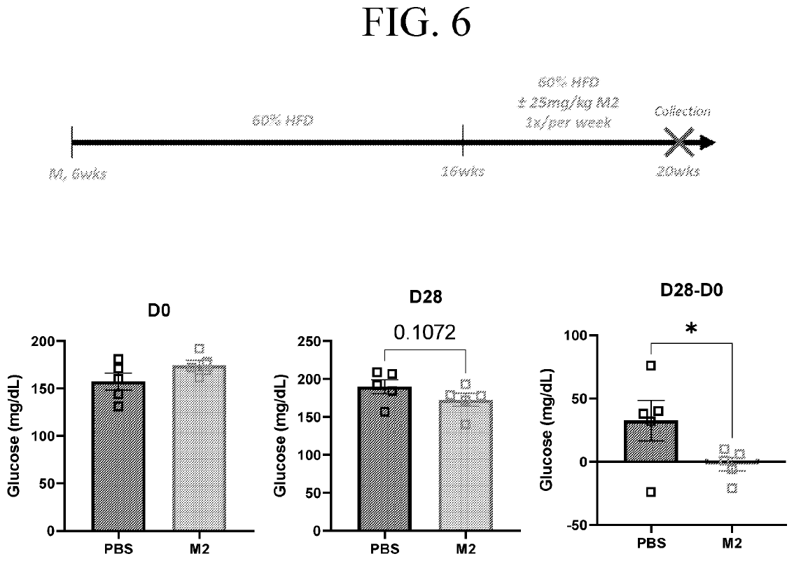

Glucose measurement. Glucose was measured using a glucometer (Contour next, Acensia, Parsippany, NJ) using blood collected from the tail vein on day 0 and day 28 of the experiment to determine changes in glucose ±M2 antibody. The results are shown in FIG. 6.

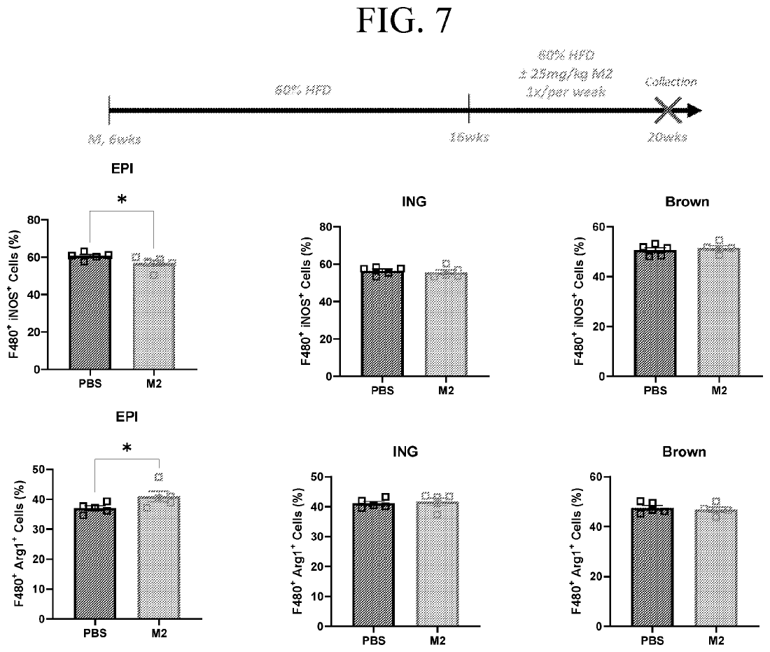

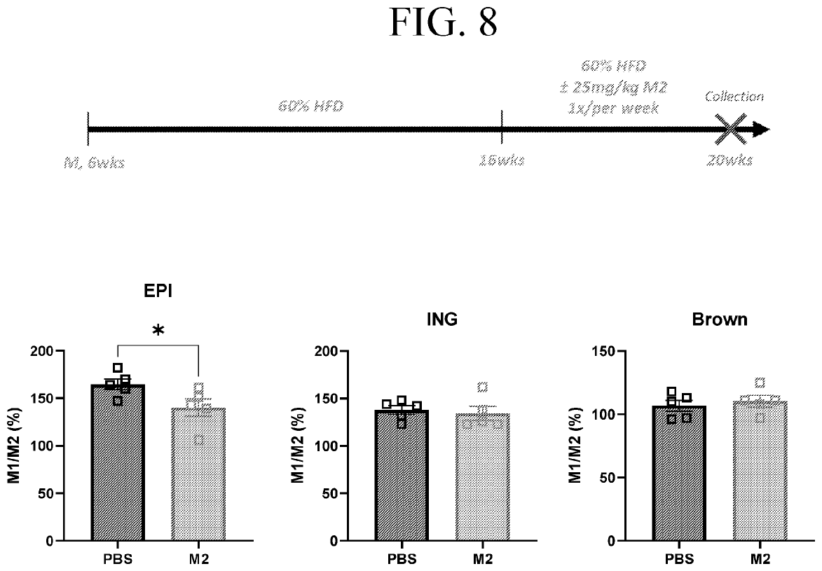

Flow cytometry adipose tissue macrophage characterization. Adipose tissue stromal vascular cells (SVCs, containing macrophages) were isolated using collagenase digestion, stained with cell surface markers, and underwent flow cytometry analysis for macrophage characterization. F480+iNOS+ population was used to identify the “M1”, pro-inflammatory phenotype. F480+Arg1+ population was used to identify the “M2”, anti-inflammatory phenotype. F480+iNOS+“M1” pro-inflammatory phenotype population (%) was divided by the F480+Arg1+“M2” anti-inflammatory phenotype population (%) to obtain the ratio. The results are shown in FIGS. 7 and 8.

Results. Anti-Cx43 hemichannel activating antibody (M2 antibody) decreases bone marrow adiposity. H&E images of bone marrow adiposity (BMA) in the trabecular bone area of WT (C57BL/6) male mice (22-months old), subjected to either PBS or M2 antibody (25 mg/kg) intraperitoneal (IP) injections for 2 weeks decreased BMA (FIG. 1).

Anti-Cx43 hemichannel activating antibody (M2 antibody) decreases weight gained as a result of a high fat diet. Weekly IP injections of an anti-Cx43 hemichannel activating antibody (M2 antibody) for 4 weeks significantly decreases weight gained (p<0.05) as a result of a high-fat diet (FIG. 2). A positive sign of metabolic health.

Anti-Cx43 hemichannel activating antibody (M2 antibody) treatment decreases percent fat gained as a result of a high fat diet. Anti-Cx43 hemichannel activating antibody (M2 antibody) treatment for 4 weeks significantly decreases the percent fat gained as a result of a high fat diet (HFD), measured through DEXA body composition analysis (FIG. 3).

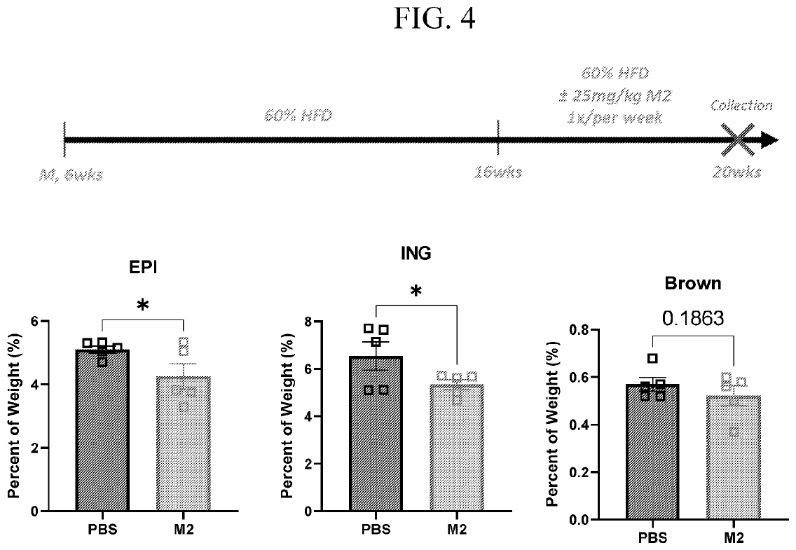

Anti-Cx43 hemichannel activating antibody (M2 antibody) treatment significantly decreases the volume of visceral and subcutaneous fat. Fat tissue weights (percent normalized to total body weight) of diet-induced obesity (DIO) WT (C57BL/6) male mice (20-weeks old) subjected to either PBS or M2 antibody (25 mg/kg) showed a significant decrease in visceral (EPI) and subcutaneous (ING) adipose tissue (FIG. 4).

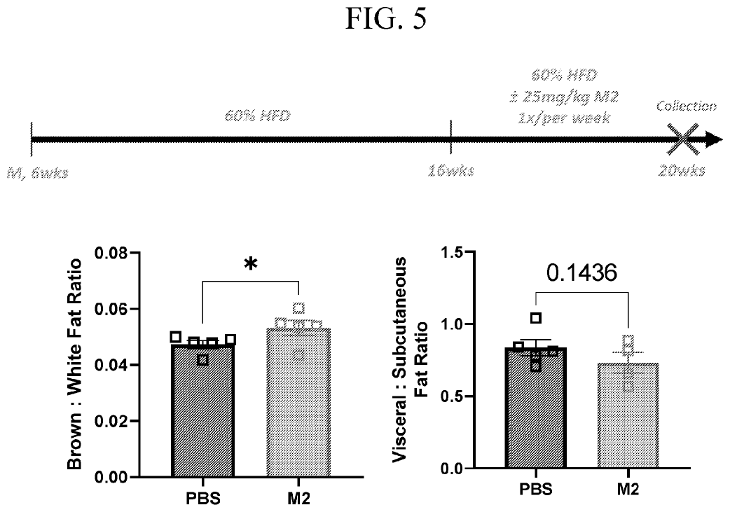

Anti-Cx43 hemichannel activating antibody (M2 antibody) treatment has a significantly higher brown:white fat ratio, and lower visceral:subcutaneous fat ratio. As a sign of better metabolic health, a significant increase in brown:white fat ratio and decrease in visceral:subcutaneous fat ratio was seen with the treatment of an anti-Cx43 hemichannel activating antibody (M2 antibody)(FIG. 5).

Anti-Cx43 hemichannel activating antibody (M2 antibody) treatment significantly decreases glucose gained as a result of a high fat diet (HFD). Non-fasted glucose levels, measured at baseline and post-4 weeks of either PBS or M2 antibody (25 mg/kg) treatment, showed a significant hindrance in glucose increase with a high-fat diet in the M2 antibody-treated mice (FIG. 6).

Anti-Cx43 hemichannel activating antibody (M2 antibody) treatment decreases pro-inflammatory macrophage buildup and increases anti-inflammatory macrophages in visceral adipose tissue. Isolation and characterization of adipose tissue macrophages shows a significant decrease in pro-inflammatory F480+iNOS+, and an increase in F480+Arg1+ anti-inflammatory macrophages in visceral (EPI) fat in M2 antibody-treated mice (FIG. 7).

Anti-Cx43 hemichannel activating antibody (M2 antibody) treatment decreases M1/M2 macrophage ratio in visceral adipose tissue. Further characterization of the macrophage landscape, in visceral adipose tissue (EPI), a significant decrease in the M1/M2 ratio was observed with M2 antibody treatment demonstrating improved or better metabolic health (FIG. 8).

Anti-Cx43 hemichannel activating antibody (M2 antibody) treatment prevents adipose tissue hypertrophy, NAFLD NASH, and brown fat white adipose tissue infiltration. H&E images of adipose tissue and liver of diet-induced obesity (DIO) WT (C57BL/6) male mice (20-weeks old) subjected to either PBS or M2 antibody (25 mg/kg) intraperitoneal (IP) injections for 4 weeks showed an overarching improvement in multiple qualitative parameters of tissue metabolic health with M2 antibody treatment (FIG. 9).

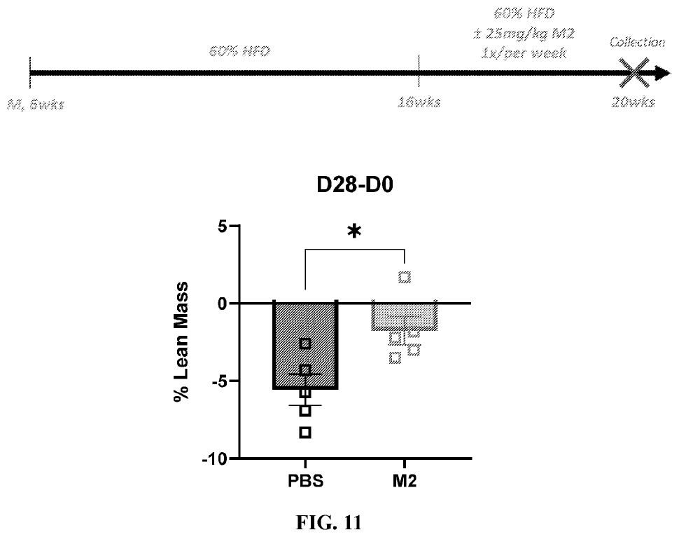

Anti-Cx43 hemichannel activating antibody (M2 antibody) treatment significantly decreases percent fat gained as a result of high fat diet (HFD) while helping retain lean skeletal muscle mass. Body composition analysis (% Lean Mass) of diet-induced obesity (DIO) WT (C57BL/6) male mice (16-weeks old, DO or 20-weeks old, D28) were subjected to either PBS or M2 (Cx43 hemichannel activating antibody, 25 mg/kg) intraperitoneal (IP) injections for 4 weeks. % Fat was determined using dual-energy X-ray absorptiometry (DEXA, PIXImus; Lunar, Madison, WI). DIO mice (60% HFD) were purchased from The Jackson Laboratory (Bar Harbor, ME) at 16-weeks of age, were allowed to acclimate and then randomly chosen for experimental groups. 60% HFD was maintained throughout the study. n=5, student's t-test, p≤0.05, results presented as mean±SEM. M2 antibody injections significantly retained % Lean Mass as a result of HFD, during the period of treatment (FIG. 11).

Together, the results provide evidence that Ocy Cx43 HCs are responsible for changes in adipose tissue formation, thus, altering Ocy Cx43 hemichannel activity using an anti-Cx43 hemichannel activating antibody (e.g., the M2 antibody) can be a useful treatment of metabolic diseases, including obesity and type II diabetes.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

We claim:

1. A method of treating or preventing obesity in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

2. A method of treating or preventing obesity in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

3. A method of treating or preventing obesity in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

4. A method of preventing or reducing fat accumulation in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

5. A method of preventing or reducing fat accumulation in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

6. A method of preventing or reducing fat accumulation in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

7. A method of reducing weight gain in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

8. A method of reducing weight gain in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

9. A method of reducing weight gain in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

10. A method of decreasing bone marrow adiposity, the method comprising administering to a subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

11. A method of decreasing bone marrow adiposity, the method comprising administering to a subject a therapeutically effective amount of anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

12. A method of decreasing bone marrow adiposity, the method comprising administering to a subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

13. A method of reducing the volume of visceral and subcutaneous fat in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

14. A method of reducing the volume of visceral and subcutaneous fat in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

15. A method of reducing the volume of visceral and subcutaneous fat in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

16. A method of reducing blood glucose levels in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

17. A method of reducing blood glucose levels in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

18. A method of reducing blood glucose levels in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

19. A method of reducing pro-inflammatory macrophage buildup in visceral adipose tissue, the method comprising administering to a subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

20. A method of reducing pro-inflammatory macrophage buildup in visceral adipose tissue, the method comprising administering to a subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

21. A method of reducing pro-inflammatory macrophage buildup in visceral adipose tissue, the method comprising administering to a subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

22. A method of increasing anti-inflammatory macrophages in visceral adipose tissue, the method comprising administering to a subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

23. A method of increasing anti-inflammatory macrophages in visceral adipose tissue, the method comprising administering to a subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

24. A method of increasing anti-inflammatory macrophages in visceral adipose tissue, the method comprising administering to a subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

25. A method of preventing or reducing adipose tissue hypertrophy in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

26. A method of preventing or reducing adipose tissue hypertrophy in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

27. A method of preventing or reducing adipose tissue hypertrophy in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

28. A method of preventing or treating nonalcoholic fatty liver disease or nonalcoholic steatohepatitis in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

29. A method of preventing or treating nonalcoholic fatty liver disease or nonalcoholic steatohepatitis in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

30. A method of preventing or treating nonalcoholic fatty liver disease or nonalcoholic steatohepatitis in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

31. A method of preventing or reducing fat accumulation and retaining or increasing lean and/or skeletal muscle mass in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

32. A method of preventing or reducing fat accumulation and retaining or increasing lean and/or skeletal muscle mass in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

33. A method of preventing or reducing fat accumulation and retaining or increasing lean and/or skeletal muscle mass in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

34. A method of reducing weight gain and retaining or increasing lean and/or skeletal muscle mass in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

35. A method of reducing weight gain and retaining or increasing lean and/or skeletal muscle mass in a subject, the methods comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

36. A method of reducing weight gain and retaining or increasing lean and/or skeletal muscle mass in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

37. A method of reducing the volume of visceral and subcutaneous fat, and retaining or increasing lean and/or skeletal muscle mass in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58.

38. A method of reducing the volume of visceral and subcutaneous fat, and retaining or increasing lean and/or skeletal muscle mass in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable light chain comprising a sequence having at least 90% identity to a sequence set forth in SEQ ID NO: 63.

39. A method of reducing the volume of visceral and subcutaneous fat, and retaining or increasing lean and/or skeletal muscle mass in a subject, the method comprising administering to the subject a therapeutically effective amount of an anti-connexin Cx43 hemichannel activating antibody or a fragment thereof, wherein the anti-connexin Cx43 hemichannel activating antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 63.

40. The method of any one of claims 1, 3, 4, 6, 7, 9, 10, 12, 13, 15, 16, 18, 19, 21, 22, 24, 25, 27, 28, 30, 31, 33, 34, 36, 37, or 39, wherein the anti-connexin Cx43 hemichannel activating antibody or fragment thereof comprises a heavy chain immunoglobulin variable region comprising:
a) a complementarity determining region 1 (CDR1) comprising the sequence of SEQ ID NO: 19 or a variant thereof;
b) a complementarity determining region 2 (CDR2) comprising the sequence of SEQ ID NO: 20 or a variant thereof, and/or
c) a complementarity determining region (CDR3) comprising the sequence of SEQ ID NO: 21 or a variant thereof.

41. The method of any one of claims 2, 3, 5, 6, 8, 9, 11, 12, 14, 15, 17, 18, 20, 21, 23, 24, 26, 27, 29, 30, 35, 36, 38, or 39, wherein the anti-connexin Cx43 hemichannel activating antibody or fragment thereof comprises a light chain immunoglobulin variable region comprising:
a) a complementarity determining region 1 (CDR1) comprising the sequence of SEQ ID NO: 49 or a variant thereof;
b) a complementarity determining region 2 (CDR2) comprising the sequence of SEQ ID NO: 50 or a variant thereof; and/or
c) a complementarity determining region 3 (CDR3) comprising the sequence of SEQ ID NO: 51 or a variant thereof.

42. The method of claim 40 or 41, wherein any one of the heavy chain CDR1, CDR2 or CDR3 or the light chain CDR1, CDR2, or CDR3 comprises at least one amino acid substitution as compared to the parent CDR.

43. The method of claim 40 or 41, wherein any one of the heavy chain CDR1, CDR2 or CDR3 variants or the light chain CDR1, CDR2 or CDR variants comprise at least one amino acid substitution as compared to the parent CDR.

44. The method of claim 42 or 43, wherein the at least one amino acid substitution is a cysteine residue to another amino acid or a glycine to another amino acid.

45. The method of claim 42 or 43, wherein the at least one amino acid substitution decreases deamidation.

46. The method of any of claims 1, 3, 4, 6, 7, 9, 10, 12, 13, 15, 16, 18, 19, 21, 22, 24, 25, 27, 28, 30, 31, 33, 34, 36, 37, or 39, wherein the anti-connexin Cx43 hemichannel activating antibody or fragment thereof comprises a heavy chain immunoglobulin variable region comprising:
a) a complementarity determining region 1 (CDR1) comprising a sequence having at least 60% identity to a sequence set forth in SEQ ID NO: 19;
b) a complementarity determining region 2 (CDR2) comprising a sequence having at least 60% identity to a sequence set forth in SEQ ID NO: 20; and/or
c) a complementarity determining region 3 (CDR3) comprising a sequence having at least 60% identity to a sequence set forth in SEQ TD NO: 21.

47. The method of any of claims 2, 3, 5, 6, 8, 9, 11, 12, 14, 15, 17, 18, 20, 21, 23, 24, 26, 27, 29, 30, 35, 36, 38, or 39, wherein the anti-connexin Cx43 hemichannel activating antibody or fragment thereof comprises a light chain immunoglobulin variable region comprising:
a) a complementarity determining region 1 (CDR1) comprising a sequence having at least 60% identity to a sequence set forth in SEQ ID NO: 49;
b) a complementarity determining region 2 (CDR2) comprising a sequence having at least 60% identity to a sequence set forth in SEQ ID NO: 50; and/or
c) a complementarity determining region 3 (CDR3) comprising a sequence having at least 60% identity to a sequence set forth in SEQ ID NO: 51.

48. The method of any of the preceding claims, comprising administering to the subject an effective amount an expression vector encoding the anti-connexin Cx43 hemichannel activating antibody.

49. The method of any of the preceding claims, wherein the anti-connexin Cx43 hemichannel activating antibody is administered in a pharmaceutically acceptable composition.

50. The method of any of the preceding claims, wherein the anti-connexin Cx43 hemichannel activating antibody is administered systemically.

51. The method of claim 50, wherein the anti-connexin Cx43 hemichannel activating antibody is administered intravenously, intradermally, intratumorally, intramuscularly, intraperitoneally, subcutaneously, or locally.

52. The method of claim 49, wherein the anti-connexin Cx43 hemichannel activating antibody is a humanized antibody.

53. The method of any of claims 1-47, further comprising administering at least a second therapeutic agent to the subject.

54. The method of claim 53, wherein the second therapeutic agent is an anti-obesity therapeutic agent or an anti-diabetes therapeutic agent.

55. The method of any of the preceding claims, wherein the anti-connexin Cx43 hemichannel activating antibody or fragment thereof binds to a Cx43 hemichannel.

56. The method of any the preceding claims, wherein the anti-connexin Cx43 hemichannel activating antibody or fragment thereof further comprises a tag sequence.

57. The method of any of the preceding claims, wherein the anti-connexin Cx43 hemichannel activating antibody or fragment thereof stimulates the opening of a Cx43 hemichannel.

58. The method of any of the preceding claims, wherein the fragment thereof is a Fab fragment, an Fab′ fragment or an F(ab′)2 fragment.

59. The method of claim 49, wherein the pharmaceutical composition is lyophilized.

60. The method of claim 3, 6, 9, 12, 15, 18, 21, 24, 27, 30, 33, 36, or 39, wherein the anti-connexin Cx43 hemichannel activating antibody or fragment thereof comprises:
a) a heavy chain immunoglobulin variable region comprising:
i) a first complementarity determining region 1 comprising a sequence having at least 60% identity to SEQ ID NO: 19;
ii) a second complementarity determining region 2 comprising a sequence having at least 60% identity to SEQ ID NO: 20; and
iii) a third complementarity determining region 3 comprising a sequence having at least 60% identity to SEQ ID NO: 21; and
b) a light chain immunoglobulin variable region comprising:
i) a first complementarity determining region 1 comprising a sequence having at least 60% identity to SEQ ID NO: 49;
ii) a second complementarity determining region 1 comprising a sequence having at least 60% identity to SEQ ID NO: 50; and
iii) a third complementarity determining region 1 comprising a sequence having at least 60% identity to SEQ ID NO: 51.

61. The method of claim 60, wherein the anti-connexin Cx43 hemichannel activating antibody is a humanized antibody.

62. The method of any of claims 1, 3, 4, 6, 7, 9, 10, 12, 13, 15, 16, 18, 19, 21, 22, 24, 25, 27, 28, 30, 31, 33, 34, 36, 37, or 39, wherein the anti-connexin Cx43 hemichannel activating antibody or fragment thereof comprises a heavy chain immunoglobulin variable region comprising:
a) a complementarity determining region 1 (CDR1) comprising a sequence having a single amino acid change compared to a sequence set forth in SEQ ID NO: 19;
b) a complementarity determining region 2 (CDR2) comprising a sequence having a single amino acid change compared to a sequence set forth in SEQ ID NO: 20; and/or
c) a complementarity determining region 3 (CDR3) comprising a sequence having a single amino acid change compared to a sequence set forth in SEQ ID NO: 21.

63. The method of any of claims 2, 3, 5, 6, 8, 9, 11, 12, 14, 15, 17 18, 20, 21, 23, 24, 26, 27, 29, 30, 35, 36, 38, or 39, wherein the anti-connexin Cx43 hemichannel activating antibody or fragment thereof comprises a light chain immunoglobulin variable region comprising:
a) a complementarity determining region 1 (CDR1) comprising a sequence having a single amino acid change compared to a sequence set forth in SEQ ID NO: 49;
b) a complementarity determining region 2 (CDR2) comprising a sequence having a single amino acid change compared to a sequence set forth in SEQ ID NO: 50; and/or
c) a complementarity determining region 3 (CDR3) comprising a sequence having a single amino acid change compared to a sequence set forth in SEQ ID NO: 51.

64. The method of claim 3, 6, 9, 12, 15, 18, 21, 24, 27, 30, 33, 36, or 39, wherein the anti-connexin Cx43 hemichannel activating antibody or fragment thereof comprises:
a) a heavy chain immunoglobulin variable region comprising:
i) a first complementarity region 1 comprising a sequence having a single amino acid change compared to SEQ ID NO: 19;
ii) a second complementarity region 2 comprising a sequence having a single amino acid change compared to SEQ ID NO: 20; and
iii) a third complementarity region 3 comprising a sequence having a single amino acid change compared to SEQ ID NO: 21; and
b) a light chain immunoglobulin variable region comprising:
i) a first complementarity region 1 comprising a sequence having a single amino acid change compared to SEQ ID NO: 49;
ii) a second complementarity region 1 comprising a sequence having a single amino acid change compared to SEQ ID NO: 50; and
iii) a third complementarity region 1 comprising a sequence having a single amino acid change compared to SEQ ID NO: 51.

65. The method of any of the preceding claims, wherein the subject is obese or has type II diabetes.
